(19) United States
(12) Patent Application Publication
(10) Pub. No.: US 2022/0313980 A1
(43) Pub. Date: Oct. 6, 2022

(54) CATHETER BLOOD PUMPS AND IMPELLERS

(71) Applicants: Daniel HILDEBRAND, Santa Cruz, CA (US); Michael CALOMENI, San Jose, CA (US); Ari RYAN, Sunnyvale, CA (US); Daniel VARGHAI, Campbell, CA (US); Janine ROBINSON, Half Moon Bay, CA (US)

(72) Inventors: Daniel HILDEBRAND, Santa Cruz, CA (US); Michael CALOMENI, San Jose, CA (US); Ari RYAN, Sunnyvale, CA (US); Daniel VARGHAI, Campbell, CA (US); Janine ROBINSON, Half Moon Bay, CA (US)

(21) Appl. No.: 17/632,550

(22) PCT Filed: Aug. 7, 2020
(86) PCT No.: PCT/US2020/045438

§ 371 Date: Feb. 3, 2022

Related U.S. Application Data
(60) Provisional application No. 62/883,728, filed on Aug. 7, 2019.

(51) Int. Cl. A61M 60/221; A61M 60/13

(52) U.S. Cl. CPC A61M 60/221; A61M 60/13

(57) ABSTRACT

Catheter blood pumps that include an expandable pump portion extending distally from a catheter. The pump portions may include an expandable impeller housing that includes an expandable blood conduit defining a blood lumen. The pump portions may include a collapsible impeller that includes a collapsible and optionally inflatable hub and one or more collapsible blades extending from the hub.

### CROSS REFERENCE TO RELATED APPLICATIONS

This applications claims priority to U.S. Prov. App. No. 62/883,728, filed Aug. 7, 2019 the disclosure of which is incorporated by reference herein for all purposes.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

Patients with heart disease can have severely compromised ability to drive blood flow through the heart and vasculature, presenting for example substantial risks during corrective procedures such as balloon angioplasty and stent delivery. There is a need for ways to improve the volume or stability of cardiac outflow for these patients, especially during corrective procedures.

Intra-aortic balloon pumps (IABP) are commonly used to support circulatory function, such as treating heart failure patients. Use of IABPs is common for treatment of heart failure patients, such as supporting a patient during high-risk percutaneous coronary intervention (HRPCI), stabilizing patient blood flow after cardiogenic shock, treating a patient associated with acute myocardial infarction (AMI) or treating decompensated heart failure. Such circulatory support may be used alone or in with pharmacological treatment.

An IABP commonly works by being placed within the aorta and being inflated and deflated in counterpulsation fashion with the heart contractions, and one of the functions is to attempt to provide additive support to the circulatory system.

More recently, minimally-invasive rotary blood pumps have been developed that can be inserted into the body in connection with the cardiovascular system, such as pumping arterial blood from the left ventricle into the aorta to add to the native blood pumping ability of the left side of the patient's heart. Another known method is to pump venous blood from the right ventricle to the pulmonary artery to add to the native blood pumping ability of the right side of the patient's heart. An overall goal is to reduce the workload on the patient's heart muscle to stabilize the patient, such as during a medical procedure that may put additional stress on the heart, to stabilize the patient prior to heart transplant, or for continuing support of the patient.

The smallest rotary blood pumps currently available can be percutaneously inserted into the vasculature of a patient through an access sheath, thereby not requiring surgical intervention, or through a vascular access graft. A description of this type of device is a percutaneously-inserted ventricular support device.

There is a need to provide additional improvements to the field of ventricular support devices and similar blood pumps for treating compromised cardiac blood flow.

### SUMMARY OF THE DISCLOSURE

An exemplary aspect of the disclosure is a catheter blood pump that includes an expandable pump portion extending distally from a catheter.

In any embodiment of this aspect, the pump portion may include an expandable impeller housing that includes an expandable blood conduit defining a blood lumen.

In any embodiment of this aspect the pump portion may include a collapsible impeller comprising a collapsible and optionally inflatable hub and one or more blades extending from the hub. A collapsible hub may have a ramped surface that is disposed at least partially proximal to a proximal end of the blood conduit. A ramped surface may be positioned relative to the proximal end of the blood conduit to create at least partial radial flow at an outflow of the pump portion. An inflatable hub may have an internal volume in fluid communication with a catheter fluid pathway to facilitate fluid delivery to the internal volume to increase fluid pressure within the internal volume of the hub.

In any embodiment of this aspect, an inflatable hub may further comprise a proximal non-bladed section, the proximal non-bladed having an outer profile with a tapered configuration that tapers downward in a proximal direction, the outer profile at least partially facilitating collapse of the inflatable hub.

In any embodiment of this aspect, a ramped surface may be in a bladed region of a collapsible hub.

In any embodiment of this aspect, an internal hub volume may be greater in a deployed configuration than in a collapsed delivery configuration.

In any embodiment of this aspect, one or more impeller blades may not be in fluid communication with the internal volume.

In any embodiment of this aspect, an impeller hub may comprise at least one material such that the impeller hub is stiffer after an inflation fluid is delivered to an inner volume of the impeller hub.

In any embodiment of this aspect, an inflatable hub may comprise a first section with a first thickness, and a second section with a second thickness different than the first thickness. A first section and a second section comprises polymeric material.

In any embodiment of this aspect, an internal volume may be adapted to be in fluid communication with a purge fluid source, the purge fluid source adapted to be disposed outside of a patient's body.

In any embodiment of this aspect, a hub may include a bladed region with a trumpet configuration, with one or more blades disposed in the bladed region.

In any embodiment of this aspect, an inflatable hub may be inflatable in a bladed region and inflatable in a non-bladed region. An internal volume may include a bladed internal volume and a non-bladed internal volume. A bladed internal volume may be greater than an internal non-bladed volume when the hub is inflated. A bladed internal volume may be less than an internal non-bladed volume when the hub is inflated.

In any embodiment of this aspect, an inflatable hub may comprise a compliant or semi-compliant material.

In any embodiment of this aspect, an inflatable hub may comprise at least one material such that it self-expands to an at least partially deployed configuration upon removal of a sheathing force. An inflatable hub may deform from a self-expanded configuration after the fluid delivery. An inflatable hub may not substantially deform from a self-expanded configuration after the fluid delivery.

In any embodiment of this aspect, at least one surface that is part of the hub is deformable from a collapsed delivery position to a fully deployed configuration in which an outermost dimension of the at least one surface, relative to a long axis of the impeller, is greater in the fully deployed configuration than it is in the collapsed delivery position. The at least one surface may be configured to at least partially self-expand, and wherein the at least one surface may be adapted to be pressurized after at least partial self-expansion upon delivery of the fluid.

A collapsible and optionally inflatable hub may comprise a plurality of support members adapted and configured to provide radial support to the hub. A plurality of support members may have an at-rest configuration toward which they are biased to return upon the removal of a sheathing force. A plurality of support members may be spaced apart circumferentially around the inflatable hub. Support members may comprise nitinol. A collapsible hub may further comprise a membrane extending between first and second of a plurality of support members, the membrane at least partially defining an hub internal volume. A plurality of support members may be coupled to the ramped surface, optionally forming part of the ramped surface. A plurality of support members, when in fully deployed configurations, may extend from a proximal rotatable member that is disposed proximally relative to the one or more blades into a bladed impeller region that includes a ramped surface. A plurality of support members, moving in a proximal direction, may increase in outer dimension in a bladed impeller region, and may decreases in outer dimension in a non-bladed impeller region.

In any embodiment of this aspect, a hub internal volume may be adapted to be in communication with a vacuum source to allow a vacuum to be pulled on the hub internal volume to facilitate fluid remove and hub collapse.

In any embodiment of this aspect, a proximal region of a bladed region of the impeller, including a ramped surface, may be configured to create a greater amount of radial flow than a distal region of the bladed region.

In any embodiment of this aspect, a hub in a bladed region gradually increases in height between a distal end of a bladed region and a proximal end of the bladed region.

In any embodiment of this aspect, a proximal non-bladed tapered hub region may be configured with at least one surface to entrain flow.

In any embodiment of this aspect, an impeller is a proximal impeller, the blood pump further comprising a distal impeller. A distal impeller may be configured as an axial flow impeller.

In any embodiment of this aspect, a collapsible impeller may be a proximal impeller, the pump portion further comprising a distal impeller, wherein the proximal and distal impellers may have different configurations with differences in or more of blade configuration, hub configuration, impeller length, blade length, outermost radial dimension, or flow type (e.g., axial vs mixed or radial). Proximal and distal impellers may be different flow type impellers, and each may be selected from the group consisting of a radial flow impeller, an axial flow impeller, and a mixed flow impeller.

In any embodiment of this aspect, an inflatable impeller may have a proximal non-bladed region with an outer surface that is configured to facilitate collapse of the impeller in response to a distally applied collapse force. A proximal non-bladed region may have a conical configuration.

In any embodiment of this aspect, an inflatable hub may comprise a proximal end region with an expandable non-bladed re-sheathing guide.

One exemplary aspect of the disclosure is a catheter blood pump that includes an expandable pump portion extending distally from a catheter. The pump portion may include an expandable impeller housing that includes an expandable blood conduit defining a blood lumen. The pump portion may also include a collapsible impeller comprising a collapsible hub and one or more blades extending from the hub, wherein the collapsible hub is at least partially self-expanding. A collapsible hub may be in fluid communication with a fluid source such that it is adapted to be inflated with fluid.

In this aspect, the pump portion may include any of the catheter blood pump features set forth in paragraphs [0009]-[0032] herein.

One exemplary aspect of the disclosure is a pump portion of a catheter blood pump that includes an expandable impeller housing that includes an expandable blood conduit defining a blood lumen. The pump portion may further include one or more collapsible impellers, the one or more collapsible impellers including one or more deformable blades and one or more support members, the one or more support members extending along an outer edge surface of one of the one or more deformable blades.

In any embodiment of this aspect, any of the one or more collapsible impellers may include a collapsible hub. The one or more deformable blades may be skinned One or more support members may comprise nitinol.

One exemplary aspect of the disclosure is a method of deploying a pump portion of a catheter blood pump. The method may include delivering a pump portion in a collapsed delivery configuration to a target location, the pump portion including an expandable housing, and an impeller having one or more blades extending from an impeller hub. The method may include deploying the pump portion from a delivery device to cause an expandable impeller housing to expand and to cause the one or more blades to self-expand. The method may include delivering fluid into the impeller to cause an increase in stiffness in the impeller hub.

### BRIEF DESCRIPTION OF THE DRAWINGS

FIG. 1 is a side view of an exemplary expandable pump portion that includes an expandable impeller housing that includes a scaffold and blood conduit, and a plurality of impellers.

FIG. 2 is a side view of an exemplary expandable pump portion that includes an expandable impeller housing, a blood conduit, a plurality of impellers, and a plurality of expandable scaffolds sections or support members.

FIGS. 3A, 3B, 3C and 3D illustrate an exemplary expandable pump portion that includes a blood conduit, a plurality of impellers, and a plurality of expandable scaffold sections or support members.

FIG. 4 illustrates an exemplary target location of an expandable pump portion, the pump portion including a blood conduit, a plurality of expandable scaffold sections or support members, and a plurality of impellers.

FIG. 5 illustrates an exemplary pump portion including an expandable impeller housing, a blood conduit, and a plurality of impellers.

FIG. 6A illustrates at least a portion of an exemplary catheter blood pump that includes a pump portion, wherein at least two different impellers can be rotated at different speeds.

FIG. 6B illustrates at least a portion of an exemplary catheter blood pump that includes a pump portion, where at least two different impellers can be rotated at different speeds.

FIG. 6C illustrates at least a portion of an exemplary catheter blood pump that includes a pump portion with at least two impellers having different pitches.

FIG. 7 illustrates a portion of an exemplary catheter blood pump that includes a pump portion.

FIG. 8 illustrates an exemplary expandable pump portion including a plurality of expandable impellers, including one or more bends formed therein between adjacent impellers.

FIG. 9 illustrates an exemplary expandable pump portion comprising a plurality of impellers and a blood conduit.

FIG. 10 illustrates an exemplary scaffold design and exemplary struts.

FIG. 11 illustrate an exemplary scaffold design and exemplary struts.

FIGS. 12A-12F illustrate an exemplary sequence of steps that may be performed to deploy an exemplary pump portion of a catheter blood pump.

FIG. 13 shows a collapsible impeller including a collapsible hub.

FIG. 14 shows a collapsible impeller including a collapsible hub.

FIG. 15 illustrates an expandable housing with a radially enlarged region between housing ends.

### DETAILED DESCRIPTION

The present disclosure is related to medical devices, systems, and methods of use and manufacture. Medical devices herein may include a distal pump portion (which may also be referred to herein as a working portion) adapted to be disposed within a physiologic vessel, wherein the distal pump portion includes one or more components that act upon fluid. For example, pump portions herein may include one or more rotating members that when rotated, can facilitate the movement of a fluid such as blood.

Any of the disclosure herein relating to an aspect of a system, device, or method of use can be incorporated with any other suitable disclosure herein. For example, a figure describing only one aspect of a device or method can be included with other embodiments even if that is not specifically stated in a description of one or both parts of the disclosure. It is thus understood that combinations of different portions of this disclosure are included herein.

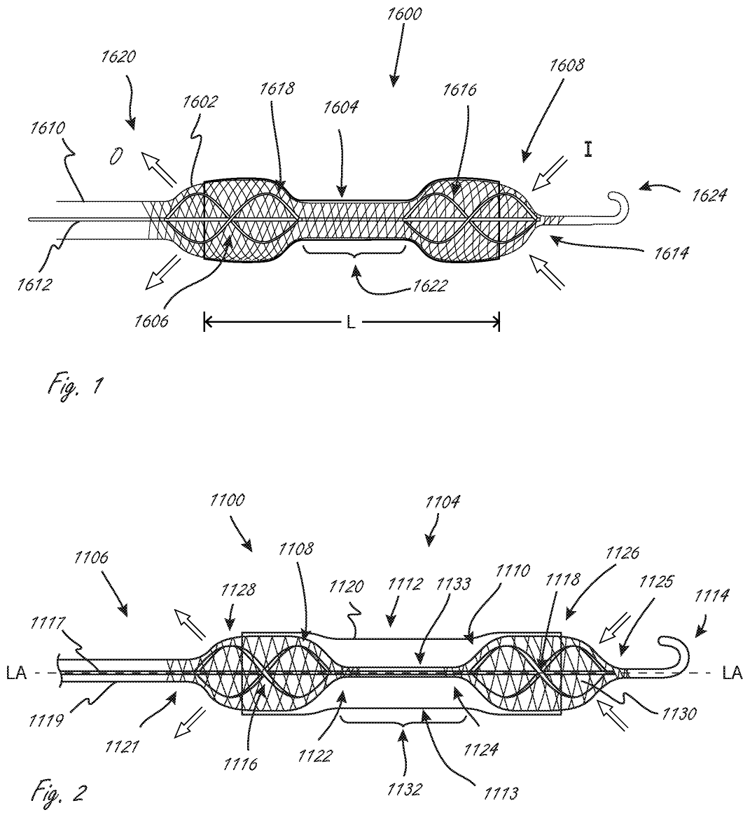

FIG. 1 is a side view illustrating a distal portion of an exemplary catheter blood pump, including pump portion 1600, wherein pump portion 1600 includes proximal impeller 1606 and distal impeller 1616, both of which are in operable communication with drive cable 1612. Pump portion 1600 is in an expanded configuration in FIG. 1, but is adapted to be collapsed to a delivery configuration so that it can be delivered with a lower profile. The impellers can be attached to drive mechanism 1612 (e.g., a drive cable). Drive mechanism 1612 is in operable communication with an external motor, not shown, and extends through elongate shaft 1610. The phrases “pump portion” and “working portion” (or derivatives thereof) may be used herein interchangeably unless indicated to the contrary. For example without limitation, “pump portion” 1600 can also be referred to herein as a “working portion.”

Pump portion 1600 also includes expandable member or expandable scaffold 1602, which in this embodiment has a proximal end 1620 that extends further proximally than a proximal end of proximal impeller 1606, and a distal end 1608 that extends further distally than a distal end 1614 of distal impeller 1616. Expandable members may also be referred to herein as expandable scaffolds or scaffold sections. Expandable scaffold 1602 is disposed radially outside of the impellers along the axial length of the impellers. Expandable scaffold 1602 can be constructed in a manner and made from materials similar to many types of expandable structures that are known in the medical arts to be able to collapsed and expanded, examples of which are provided herein. Examples of suitable materials include, but are not limited to, polyurethane, polyurethane elastomers, metallic alloys, etc.

Pump portion 1600 also includes blood conduit 1604, which is coupled to and supported by expandable member 1602, has a length L, and extends axially between the impellers. Conduit 1604 creates and provides a fluid lumen between the two impellers. When in use, fluid moves through the lumen defined by conduit 1604. The conduits herein may be non-permeable, or they may be semi-permeable, or even porous as long as they still define a lumen. The conduits herein are also flexible, unless otherwise indicated. The conduits herein extend completely around (i.e., 360 degrees) at least a portion of the pump portion. In pump portion 1600, the conduit extends completely around expandable member 1602, but does not extend all the way to the proximal end 1602 or distal end 1608 of expandable member 1602. The structure of the expandable member creates at least one inlet aperture to allow for inflow “I,” and at least one outflow aperture to allow for outflow “0.” Conduit 1604 improves impeller pumping dynamics, compared to pump portions without a conduit. As described herein, expandable members or scaffolds may also be considered to be a part of the blood conduit generally, which together define a blood lumen. In these instances the scaffold and material supported by the scaffold may be referred to herein as an expandable impeller housing or housing.

Expandable member 1602 may have a variety of constructions, and made from a variety of materials. For example, expandable member 1602 may be formed similar to expandable stents or stent-like devices, or any other example provided herein. For example without limitation, expandable member 1602 could have an open-braided construction, such as a 24-end braid, although more or fewer braid wires could be used. Exemplary materials for the expandable member as well as the struts herein include nitinol, cobalt alloys, and polymers, although other materials could be used. Expandable member 1602 has an expanded configuration, as shown, in which the outer dimension (measured orthogonally relative a longitudinal axis of the working portion) of the expandable member is greater in at least a region where it is disposed radially outside of the impellers than in a central region 1622 of the expandable member that extends axially between the impeller. Drive mechanism 1612 is co-axial with the longitudinal axis in this embodiment. In use, the central region can be placed across a valve, such as an aortic valve. In some embodiments, expandable member 1602 is adapted and constructed to expand to an outermost dimension of 12-24 F (4.0-8.0 mm) where the impellers are axially within the expandable member, and to an outermost dimension of 10-20 F (3.3-6.7 mm) in central region 1622 between the impellers. The smaller central region outer dimension can reduce forces acting on the valve, which can reduce or minimize damage to the valve. The larger dimensions of the expandable member in the regions of the impellers can help stabilize the working portion axially when in use. Expandable member 1602 has a general dumbbell configuration. Expandable member 1602 has an outer configuration that tapers as it transitions from the impeller regions to central region 1622, and again tapers at the distal and proximal ends of expandable member 1602.

Expandable member 1602 has a proximal end 1620 that is coupled to shaft 1610, and a distal end 1608 that is coupled to distal tip 1624. The impellers and drive mechanism 1612 rotate within the expandable member and conduit assembly. Drive mechanism 1612 is axially stabilized with respect to distal tip 1624, but is free to rotate with respect to tip 1624.

In some embodiments, expandable member 1602 can be collapsed by pulling tension from end-to-end on the expandable member. This may include linear motion (such as, for example without limitation, 5-20 mm of travel) to axially extend expandable member 1602 to a collapsed configuration with collapsed outer dimension(s). Expandable member 1602 can also be collapsed by pushing an outer shaft such as a sheath over the expandable member/conduit assembly, causing the expandable member and conduit to collapse towards their collapsed delivery configuration.

Impellers 1606 and 1616 are also adapted and constructed such that one or more blades will stretch or radially compress to a reduced outermost dimension (measured orthogonally to the longitudinal axis of the working portion). For example without limitation, any of the impellers herein can include one or more blades made from a plastic formulation with spring characteristics, such as any of the impellers described in U.S. Pat. No. 7,393,181, the disclosure of which is incorporated by reference herein for all purposes and can be incorporated into embodiments herein unless this disclosure indicates to the contrary. Alternatively, for example, one or more collapsible impellers can comprise a superelastic wire frame, with polymer or other material that acts as a webbing across the wire frame, such as those described in U.S. Pat. No. 6,533,716, the disclosure of which is incorporated by reference herein for all purposes.

The inflow and/or outflow configurations of working portion 1600 can be mostly axial in nature.

Exemplary sheathing and unsheathing techniques and concepts to collapse and expand medical devices are known, such as, for example, those described and shown in U.S. Pat. No. 7,841,976 or 8,052,749, the disclosures of which are incorporated by reference herein.

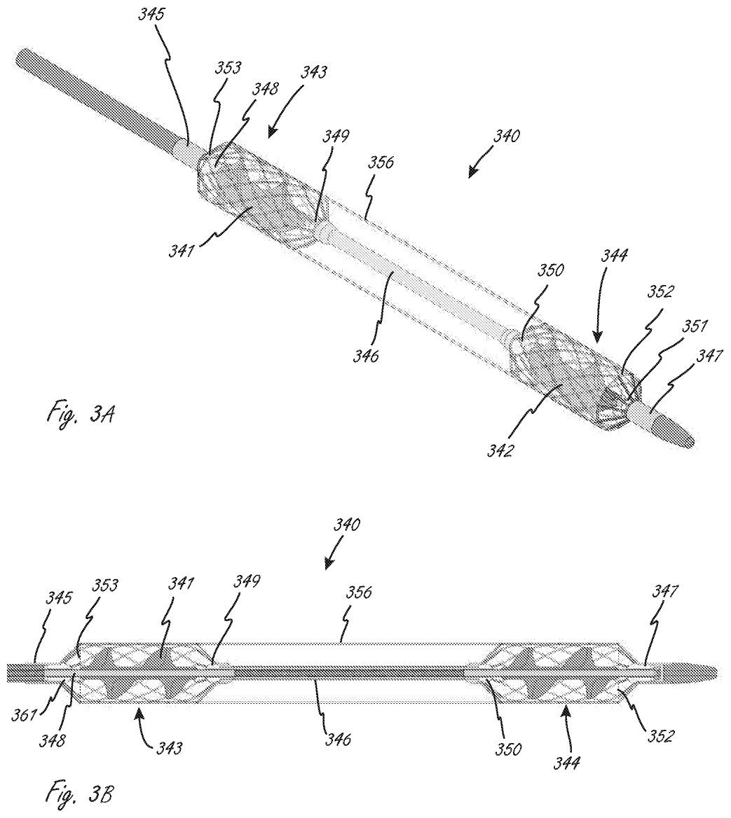

FIG. 2 is a side view illustrating a deployed configuration (shown extracorporally) of a distal portion of an exemplary embodiment of a catheter blood pump. Exemplary blood pump 1100 includes working portion 1104 (which as set forth herein may also be referred to herein as a pump portion) and an elongate portion 1106 extending from working portion 1104. Elongate portion 1106 can extend to a more proximal region of the system, not shown for clarity, and that can include, for example, a motor. Working portion 1104 includes first expandable scaffold or member 1108 and second expandable scaffold or member 1110, axially spaced apart along a longitudinal axis LA of working portion 1104. First scaffold 1108 and second scaffold 1110 (and any other separate scaffolds herein) may also be referenced as part of a common scaffold and referred to herein as scaffold sections. Spaced axially in this context refers to the entire first expandable member being axially spaced from the entire second expandable member along a longitudinal axis LA of working portion 1104. A first end 1122 of first expandable member 1108 is axially spaced from a first end 1124 of second expandable member 1110.

First and second expandable members 1108 and 1110 generally each include a plurality of elongate segments disposed relative to one another to define a plurality of apertures 1130, only one of which is labeled in the second expandable member 1110. The expandable members can have a wide variety of configurations and can be constructed in a wide variety of ways, such as any of the configurations or constructions in, for example without limitation, U.S. Pat. No. 7,841,976, or the tube in 6,533,716, which is described as a self-expanding metal endoprosthetic material. For example, without limitation, one or both of the expandable members can have a braided construction or can be at least partially formed by laser cutting a tubular element.

Working portion 1104 also includes blood conduit 1112 that is coupled to first expandable member 1108 and to second expandable member 1110, and extends axially in between first expandable member 1108 and second expandable member 1110 in the deployed configuration. A central region 1113 of conduit 1112 spans an axial distance 1132 where the working portion is void of first and second expandable members 1108 and 1110. Central region 1113 can be considered to be axially in between the expandable members. Distal end 1126 of conduit 1112 does not extend as far distally as a distal end 1125 of second expandable member 1110, and proximal end of conduit 1128 does not extend as far proximally as proximal end 1121 of first expandable member 1108.

When the disclosure herein refers to a blood conduit being coupled to an expandable scaffold or member, the term coupled in this context does not require that the conduit be directly attached to the expandable member so that conduit physically contacts the expandable member. Even if not directly attached, however, the term coupled in this context refers to the conduit and the expandable member being joined together such that as the expandable member expands or collapses, the conduit also begins to transition to a different configuration and/or size. Coupled in this context therefore refers to conduits that will move when the expandable member to which it is coupled transitions between expanded and collapsed configurations.

Any of the blood conduits herein can be deformable to some extent. For example, conduit 1112 includes elongate member 1120 that can be made of one or more materials that allow the central region 1113 of conduit to deform to some extent radially inward (towards LA) in response to, for example and when in use, forces from valve tissue (e.g., leaflets) or a replacement valve as working portion 1104 is deployed towards the configuration shown in FIG. 2. The conduit may be stretched tightly between the expandable members in some embodiments. The conduit may alternatively be designed with a looseness that causes a greater degree of compliance. This can be desirable when the working portion is disposed across fragile structures such as an aortic valve, which may allow the valve to compress the conduit in a way that minimizes point stresses in the valve. In some embodiments, the conduit may include a membrane attached to the proximal and distal expandable members. Exemplary materials that can be used for any conduits herein include, without limitations, polyurethane rubber, silicone rubber, acrylic rubber, expanded polytetrafluoroethylene, polyethylene, polyethylene terephthalate, including any combination thereof.

Any of the conduits herein can have a thickness of, for example, 0.5-20 thousandths of an inch (thou), such as 1-15 thou, or 1.5 to 15 thou, 1.5 to 10 thou, or 2 to 10 thou.

Any of the blood conduits herein, or at least a portion of the conduit, can be impermeable to blood. In FIG. 2, working portion 1104 includes a lumen that extends from distal end 1126 of conduit 1112 and extends to proximal end 1128 of conduit 1112. The lumen is defined by conduit 1112 in central region 1113, but can be thought of being defined by both the conduit and portions of the expandable members in regions axially adjacent to central region 1113. In this embodiment, however, it is the conduit material that causes the lumen to exist and prevents blood from passing through the conduit.

Any of the conduits herein that are secured to one or more expandable members can be, unless indicated to the contrary, secured so that the conduit is disposed radially outside of one or more expandable members, radially inside of one or more expandable members, or both, and the expandable member can be impregnated with the conduit material.

The proximal and distal expandable scaffolds or members help maintain the blood conduit in an open configuration to create the lumen, while each also creates a working environment for an impeller, described below. Each of the expandable scaffolds, when in the deployed configuration, is maintained in a spaced relationship relative to a respective impeller, which allows the impeller to rotate within the expandable member without contacting the expandable member. Working portion 1104 includes first impeller 1116 and second impeller 1118, with first impeller 1116 disposed radially within first expandable member 1108 and second impeller 1118 disposed radially within second expandable member 1110. In this embodiment, the two impellers even though they are distinct and separate impellers, are in operable communication with a common drive mechanism (e.g., drive cable 1117), such that when the drive mechanism is activated the two impellers rotate together. In this deployed configuration, impellers 1116 and 1118 are axially spaced apart along longitudinal axis LA, just as are the expandable members 1108 and 1110 are axially spaced apart.

Impellers 1116 and 1118 are also axially within the ends of expandable members 1108 and 1110, respectively (in addition to being radially within expandable members 1108 and 1110). The impellers herein can be considered to be axially within an expandable member even if the expandable member includes struts extending from a central region of the expandable member towards a longitudinal axis of the working portion (e.g., tapering struts in a side view). In FIG. 2, second expandable member 1110 extends from first end 1124 (proximal end) to second end 1125 (distal end).

In FIG. 2, a distal portion of impeller 1118 extends distally beyond distal end 1126 of conduit 1112, and a proximal portion of impeller 1116 extends proximally beyond proximal end 1128 of conduit 1112. In this figure, portions of each impeller are axially within the conduit in this deployed configuration.

In the exemplary embodiment shown in FIG. 2, impellers 1116 and 1118 are in operable communication with a common drive mechanism 1117, and in this embodiment, the impellers are each coupled to drive mechanism 1117, which extends through shaft 1119 and working portion 1104. Drive mechanism 1117 can be, for example, an elongate drive cable, which when rotated causes the impellers to rotate. In this example, as shown, drive mechanism 1117 extends to and is axially fixed relative to distal tip 1114, although it is adapted to rotate relative to distal tip 1114 when actuated. Thus, in this embodiment, the impellers and drive mechanism 1117 rotate together when the drive mechanism is rotated. Any number of known mechanisms can be used to rotate drive mechanism, such as with a motor (e.g., an external motor).

The expandable members and the conduit are not in rotational operable communication with the impellers and the drive mechanism. In this embodiment, proximal end 1121 of proximal expandable member 1108 is coupled to shaft 1119, which may be a shaft of elongate portion 1106 (e.g., an outer catheter shaft). Distal end 1122 of proximal expandable member 1108 is coupled to central tubular member 1133, through which drive mechanism 1117 extends. Central tubular member 1133 extends distally from proximal expandable member 1108 within conduit 1112 and is also coupled to proximal end 1124 of distal expandable member 1110. Drive mechanism 1117 thus rotates within and relative to central tubular member 1133. Central tubular member 1133 extends axially from proximal expandable member 1108 to distal expandable member 1110. Distal end 1125 of distal expandable member 1110 is coupled to distal tip 1114, as shown. Drive mechanism 1117 is adapted to rotate relative to tip 1114, but is axially fixed relative to tip 1114.

Working portion 1104 is adapted and configured to be collapsed to a smaller profile than its deployed configuration (which is shown in FIG. 2). This allows it to be delivered using a lower profile delivery device (smaller French size) than would be required if none of working portion 1104 was collapsible. Even if not specifically stated herein, any of the expandable members and impellers may be adapted and configured to be collapsible to some extent to a smaller delivery configuration.

The working portions herein can be collapsed to a collapsed delivery configuration using conventional techniques, such as with an outer sheath that is movable relative to the working portion (e.g., by axially moving one or both of the sheath and working portion). For example without limitation, any of the systems, devices, or methods shown in the following references may be used to facilitate the collapse of a working portions herein: U.S. Pat. No. 7,841,976 or 8,052,749, the disclosures of which are incorporated by reference herein for all purposes.

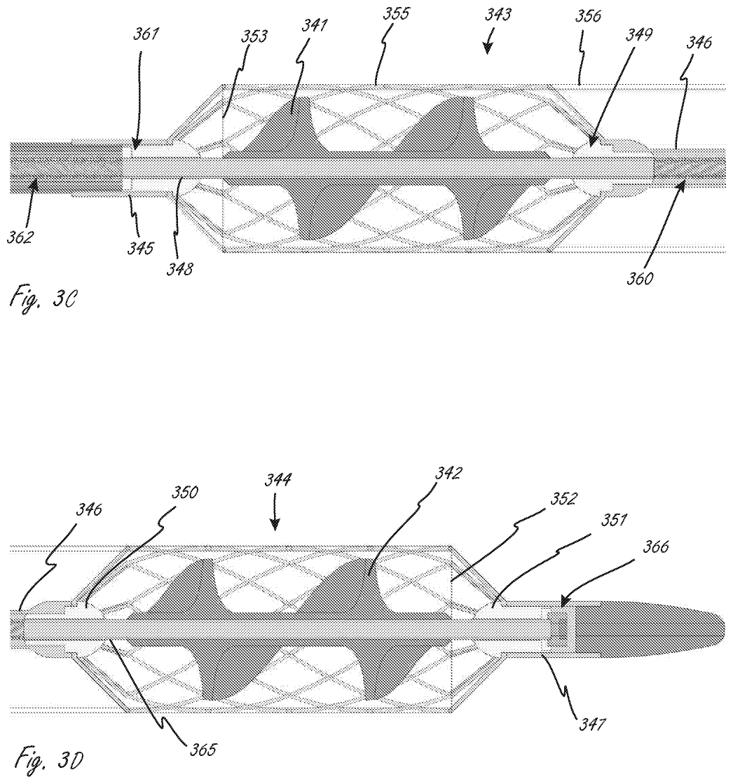

FIGS. 3A-3D show an exemplary pump portion that is similar in some ways to the pump portion shown in FIG. 2. Pump portion 340 is similar to pump portion 1104 in that in includes two expandable members axially spaced from one another when the pump portion is expanded, and a conduit extending between the two expandable members. FIG. 3A is a perspective view, FIG. 3B is a side sectional view, and FIGS. 3C and 3D are close-up side sectional views of sections of the view in FIG. 3B.

Pump portion 340 includes proximal impeller 341 and distal impeller 342, which are coupled to and in operational communication with a drive cable, which defines therein a lumen. The lumen can be sized to accommodate a guidewire, which can be used for delivery of the working portion to the desired location. The drive cable, in this embodiment, includes first section 362 (e.g., wound material), second section 348 (e.g., tubular member) to which proximal impeller 341 is coupled, third section 360 (e.g., wound material), and fourth section 365 (e.g., tubular material) to which distal impeller 342 is coupled. The drive cable sections all have the same inner diameter, so that lumen has a constant inner diameter. The drive cable sections can be secured to each other using known attachment techniques. A distal end of fourth section 365 extends to a distal region of the working portion, allowing the working portion to be, for example, advanced over a guidewire for positioning the working portion. In this embodiment the second and fourth sections can be stiffer than first and third sections. For example, second and fourth can be tubular and first and third sections can be wound material to impart less stiffness.

Pump portion 340 includes proximal expandable scaffold 343 and distal expandable scaffold 344, each of which extends radially outside of one of the impellers. The expandable scaffolds have distal and proximal ends that also extend axially beyond distal and proximal ends of the impellers, which can be seen in FIGS. 3B-3D. Coupled to the two expandable scaffolds is blood conduit 356, which has a proximal end 353 and a distal end 352. The two expandable scaffolds each include a plurality of proximal struts and a plurality of distal struts. The proximal struts in proximal expandable scaffold 343 extend to and are secured to shaft section 345, which is coupled to bearing 361, through which the drive cable extends and is configured and sized to rotate. The distal struts of proximal expandable scaffold 343 extend to and are secured to a proximal region (to a proximal end in this case) of central tubular member 346, which is disposed axially in between the expandable members. The proximal end of central tubular member 346 is coupled to bearing 349, as shown in FIG. 3C, through which the drive cable extends and rotates. The proximal struts extend axially from distal expandable scaffold 344 to and are secured to a distal region (to a distal end in this case) of central tubular member 346. Bearing 350 is also coupled to the distal region of central tubular member 346, as is shown in FIG. 3D. The drive cable extends through and rotates relative to bearing 350. Distal struts extend from the distal expandable scaffold extend to and are secured to shaft section 347 (see FIG. 3A), which can be considered part of the distal tip. Shaft section 347 is coupled to bearing 351 (see FIG. 3D), through which the drive cable extends and rotates relative to. The distal tip also includes bearing 366 (see FIG. 3D), which can be a thrust bearing. Working portion 340 can be similar to or the same in some aspects to working portion 1104, even if not explicitly included in the description. In this embodiment, conduit 356 extends at least as far as ends of the impeller, unlike in working portion 1104. Either embodiment can be modified so that the conduit extends to a position as set forth in the other embodiment. In some embodiments, section 360 can be a tubular section instead of wound.

In alternative embodiments, at least a portion of any of the impellers herein may extend outside of the fluid lumen. For example, only a portion of an impeller may extend beyond an end of the fluid lumen in either the proximal or distal direction. In some embodiments, a portion of an impeller that extends outside of the fluid lumen is a proximal portion of the impeller, and includes a proximal end (e.g., see the proximal impeller in FIG. 2). In some embodiments, the portion of the impeller that extends outside of the fluid lumen is a distal portion of the impeller, and includes a distal end (e.g., see the distal impeller in FIG. 2). When the disclosure herein refers to impellers that extend outside of the fluid lumen (or beyond an end), it is meant to refer to relative axial positions of the components, which can be most easily seen in side views or top views, such as in FIG. 2.

A second impeller at another end of the fluid lumen may not, however, extend beyond the fluid lumen. For example, an illustrative alternative design can include a proximal impeller that extends proximally beyond a proximal end of the fluid lumen (like the proximal impeller in FIG. 2), and the fluid lumen does not extend distally beyond a distal end of a distal impeller (like in FIG. 3B). Alternatively, a distal end of a distal impeller can extend distally beyond a distal end of the fluid lumen, but a proximal end of a proximal impeller does not extend proximally beyond a proximal end of the fluid lumen. In any of the pump portions herein, none of the impellers may extend beyond ends of the fluid lumen.

While specific exemplary locations may be shown herein, the fluid pumps may be able to be used in a variety of locations within a body. Some exemplary locations for placement include placement in the vicinity of an aortic valve or pulmonary valve, such as spanning the valve and positioned on one or both sides of the valve, and in the case of an aortic valve, optionally including a portion positioned in the ascending aorta. In some other embodiments, for example, the pumps may be, in use, positioned further downstream, such as being disposed in a descending aorta.

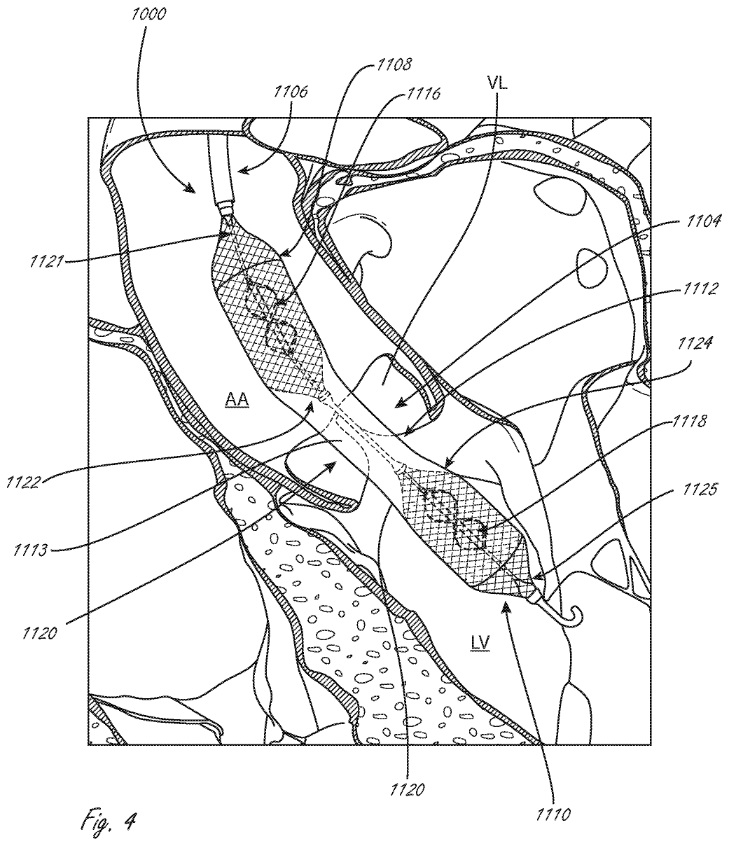

FIG. 4 illustrates an exemplary placement of pump portion 1104 from catheter blood pump 1000 from FIG. 2. Once difference shown in FIG. 4 is that the conduit extends at least as far as the ends of the impellers, like in FIGS. 3A-3D. FIG. 4 shows pump portion 1104 in a deployed configuration, positioned in place across an aortic valve. Pump portion 1104 can be delivered as shown via, for example without limitation, femoral artery access (a known access procedure). While not shown for clarity, system 1000 can also include an outer sheath or shaft in which working portion 1104 is disposed during delivery to a location near an aortic valve. The sheath or shaft can be moved proximally (towards the ascending aorta “AA” and away from left ventricle “LV”) to allow for deployment and expansion of working portion 1104. For example, the sheath can be withdrawn to allow for expansion of second expandable scaffold 1110, with continued proximal movement allowing first expandable scaffold 1108 to expand.

In this embodiment, second expandable scaffold 1110 has been expanded and positioned in a deployed configuration such that distal end 1125 is in the left ventricle “LV,” and distal to aortic valve leaflets “VL,” as well as distal to the annulus. Proximal end 1124 has also been positioned distal to leaflets VL, but in some methods proximal end 1124 may extend slightly axially within the leaflets VL. This embodiment is an example of a method in which at least half of the second expandable member 1110 is within the left ventricle, as measured along its length (measured along the longitudinal axis). And as shown, this is also an example of a method in which the entire second expandable member 1110 is within the left ventricle. This is also an example of a method in which at least half of second impeller 1118 is positioned within the left ventricle, and also an embodiment in which the entire second impeller 1118 is positioned within the left ventricle.

Continued retraction of an outer shaft or sheath (and/or distal movement of working end 1104 relative to an outer sheath or shaft) continues to release conduit 1112, until central region 1113 is released and deployed. The expansion of expandable scaffolds 1108 and 1110 causes blood conduit 1112 to assume a more open configuration, as shown in FIG. 4. Thus, while in this embodiment conduit 1112 does not have the same self-expanding properties as the expandable scaffolds, the conduit will assume a deployed, more open configuration when the working end is deployed. At least a portion of central region 1113 of conduit 1112 is positioned at an aortic valve coaptation region and engages leaflets. In FIG. 3, there is a short length of central region 1113 that extends distally beyond the leaflets VL, but at least some portion of central region 1113 is axially within the leaflets.

Continued retraction of an outer shaft or sheath (and/or distal movement of working end 1104 relative to an outer sheath or shaft) deploys first expandable member 1108. In this embodiment, first expandable scaffold 1108 has been expanded and positioned (as shown) in a deployed configuration such that proximal end 1121 is in the ascending aorta AA, and proximal to leaflets “VL.” Distal end 1122 has also been positioned proximal to leaflets VL, but in some methods distal end 1122 may extend slightly axially within the leaflets VL. This embodiment is an example of a method in which at least half of first expandable member 1110 is within the ascending aorta, as measured along its length (measured along the longitudinal axis). And as shown, this is also an example of a method in which the entire first expandable member 1110 is within the AA. This is also an example of a method in which at least half of first impeller 1116 is positioned within the AA, and also an embodiment in which the entire first impeller 1116 is positioned within the AA.

At any time during or after deployment of pump portion 1104, the position of the pump portion can be assessed in any way, such as under fluoroscopy. The position of the pump portion can be adjusted at any time during or after deployment. For example, after second expandable scaffold 1110 is released but before first expandable member 1108 is released, pump portion 1104 can be moved axially (distally or proximally) to reposition the pump portion. Additionally, for example, the pump portion can be repositioned after the entire working portion has been released from a sheath to a desired final position.

It is understood that the positions of the components (relative to the anatomy) shown in FIG. 4 are considered exemplary final positions for the different components of working portion 1104, even if there was repositioning that occurred after initial deployment.

The one or more expandable members herein can be configured to be, and can be expanded in a variety of ways, such as via self-expansion, mechanical actuation (e.g., one or more axially directed forces on the expandable member, expanded with a separate balloon positioned radially within the expandable member and inflated to push radially outward on the expandable member), or a combination thereof.

Expansion as used herein refers generally to reconfiguration to a larger profile with a larger radially outermost dimension (relative to the longitudinal axis), regardless of the specific manner in which the one or more components are expanded. For example, a stent that self-expands and/or is subject to a radially outward force can “expand” as that term is used herein. A device that unfurls or unrolls can also assume a larger profile, and can be considered to expand as that term is used herein.

The impellers can similarly be adapted and configured to be, and can be expanded in a variety of ways depending on their construction. For examples, one or more impellers can, upon release from a sheath, automatically revert to or towards a different larger profile configuration due to the material(s) and/or construction of the impeller design (see, for example, U.S. Pat. No. 6,533,716, or U.S. Pat. No. 7,393,181, both of which are incorporated by reference herein for all purposes). Retraction of an outer restraint can thus, in some embodiments, allow both the expandable member and the impeller to revert naturally to a larger profile, deployed configuration without any further actuation.

As shown in the example in FIG. 4, the working portion includes first and second impellers that are spaced on either side of an aortic valve, each disposed within a separate expandable member. This is in contrast to some designs in which a working portion includes a single elongate expandable member. Rather than a single generally tubular expandable member extending all the way across the valve, working end 1104 includes a conduit 1112 extending between expandable members 1108 and 1110. The conduit is more flexible and deformable than the expandable baskets, which can allow for more deformation of the working portion at the location of the leaflets than would occur if an expandable member spanned the aortic valve leaflets. This can cause less damage to the leaflets after the working portion has been deployed in the subject.

Additionally, forces on a central region of a single expandable member from the leaflets might translate axially to other regions of the expandable member, perhaps causing undesired deformation of the expandable member at the locations of the one or more impellers. This may cause the outer expandable member to contact the impeller, undesirably interfering with the rotation of the impeller. Designs that include separate expandable members around each impeller, particularly where each expandable member and each impeller are supported at both ends (i.e., distal and proximal), result in a high level of precision in locating the impeller relative to the expandable member. Two separate expandable members may be able to more reliably retain their deployed configurations compared with a single expandable member.

As described herein above, it may be desirable to be able to reconfigure the working portion so that it can be delivered within a 9 F sheath and still obtain high enough flow rates when in use, which is not possible with some products currently in development and/or testing. For example, some products are too large to be able to be reconfigured to a small enough delivery profile, while some smaller designs may not be able to achieve the desired high flow rates. An exemplary advantage of the examples in FIGS. 1, 2, 3A-3D and 4 is that, for example, the first and second impellers can work together to achieve the desired flow rates, and by having two axially spaced impellers, the overall working portion can be reconfigured to a smaller delivery profile than designs in which a single impeller is used to achieved the desired flow rates. These embodiments thus use a plurality of smaller, reconfigurable impellers that are axially spaced to achieve both the desired smaller delivery profile as well as to achieve the desired high flow rates.

The embodiment herein can thus achieve a smaller delivery profile while maintaining sufficiently high flow rates, while creating a more deformable and flexible central region of the working portion, the exemplary benefits of which are described above (e.g., interfacing with delicate valve leaflets).

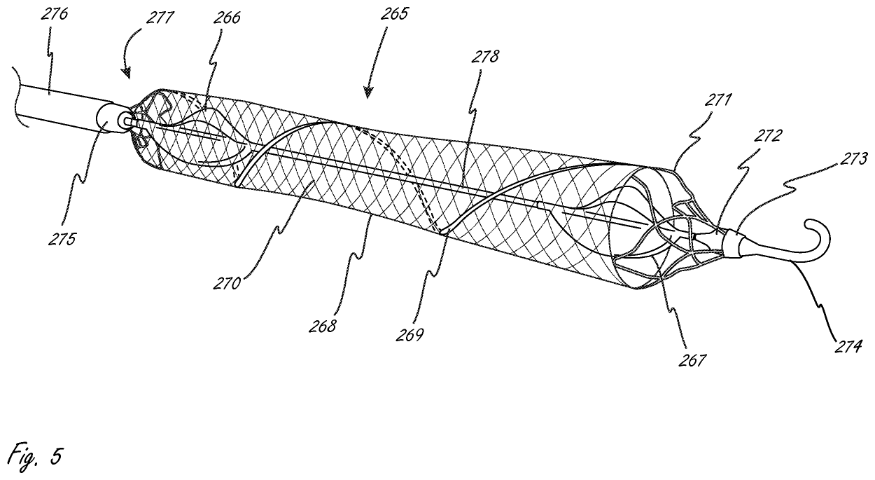

FIG. 5 illustrates a working portion that is similar to the working portion shown in FIG. 1. Working portion 265 includes proximal impeller 266, distal impeller 267, both of which are coupled to drive shaft 278, which extends into distal bearing housing 272. There is a similar proximal bearing housing at the proximal end of the working portion. Working portion also includes expandable scaffold or member, referred to 270 generally, and blood conduit 268 that is secured to the expandable member and extends almost the entire length of expandable member. Expandable member 270 includes distal struts 271 that extend to and are secured to strut support 273, which is secured to distal tip 273. Expandable member 270 also includes proximal struts there are secured to a proximal strut support. All features similar to that shown in FIG. 1 are incorporated by reference for all purposes into this embodiment even if not explicitly stated. Expandable member 265 also includes helical tension member 269 that is disposed along the periphery of the expandable member, and has a helical configuration when the expandable member is in the expanded configuration as shown. The helical tension member 269 is disposed and adapted to induce rotation wrap upon collapse. Working portion 265 can be collapsed from the shown expanded configuration while simultaneously rotating one or both impellers at a relatively slow speed to facilitate curled collapse of the impellers due to interaction with the expandable member. Helical tension member 269 (or a helical arrangement of expandable member cells) will act as a collective tension member and is configured so that when the expandable basket is pulled in tension along its length to collapse (such as by stretching to a much greater length, such as approximately doubling in length) tension member 269 is pulled into a straighter alignment, which causes rotation/twisting of the desired segment(s) of the expandable member during collapse, which causes the impeller blades to wrap radially inward as the expandable member and blades collapse. An exemplary configuration of such a tension member would have a curvilinear configuration when in helical form that is approximately equal to the maximum length of the expandable member when collapsed. In alternative embodiments, only the portion(s) of the expandable member that encloses a collapsible impeller is caused to rotate upon collapse.

There are alternative ways to construct the working portion to cause rotation of the expandable member upon collapse by elongation (and thus cause wrapping and collapse of the impeller blades). Any expandable member can be constructed with this feature, even in dual-impeller designs. For example, with an expandable member that includes a plurality of “cells,” as that term is commonly known (e.g., a laser cut elongate member), the expandable member may have a plurality of particular cells that together define a particular configuration such as a helical configuration, wherein the cells that define the configuration have different physical characteristics than other cells in the expandable member. In some embodiments the expandable member can have a braided construction, and the twist region may constitute the entire group of wires, or a significant portion (e.g., more than half), of the braided wires. Such a twisted braid construction may be accomplished, for example, during the braiding process, such as by twisting the mandrel that the wires are braided onto as the mandrel is pulled along, especially along the length of the largest-diameter portion of the braided structure. The construction could also be accomplished during a second operation of the construction process, such as mechanically twisting a braided structure prior to heat-setting the wound profile over a shaped mandrel.

Any of the blood conduits herein act to, are configured to, and are made of material(s) that create a fluid lumen therein between a first end (e.g., distal end) and a second end (e.g., proximal end). Fluid flows into the inflow region, through the fluid lumen, and then out of an outflow region. Flow into the inflow region may be labeled herein as “I,” and flow out at the outflow region may be labeled “0.” Any of the conduits herein can be impermeable. Any of the conduits herein can alternatively be semipermeable. Any of the conduits herein may also be porous, but will still define a fluid lumen therethrough. In some embodiments the conduit is a membrane, or other relatively thin layered member. Any of the conduits herein, unless indicated to the contrary, can be secured to an expandable member such that the conduit, where is it secured, can be radially inside and/or outside of the expandable member. For example, a conduit may extend radially within the expandable member so that inner surface of the conduit is radially within the expandable member where it is secured to the expandable member.

Any of the expandable scaffolds or member(s) herein may be constructed of a variety of materials and in a variety of ways. For example, the expandable member may have a braided construction, or it can be formed by laser machining. The material can be deformable, such as nitinol. The expandable member can be self-expanding or can be adapted to be at least partially actively expanded.

In some embodiments, the expandable scaffold or member is adapted to self-expand when released from within a containing tubular member such as a delivery catheter, a guide catheter or an access sheath. In some alternative embodiments, the expandable member is adapted to expand by active expansion, such as action of a pull-rod that moves at least one of the distal end and the proximal end of the expandable member toward each other. In alternative embodiments, the deployed configuration can be influenced by the configuration of one or more expandable structures. In some embodiments, the one or more expandable members can deployed, at least in part, through the influence of blood flowing through the conduit. Any combination of the above mechanisms of expansion may be used.

The blood pumps and fluid movement devices, system and methods herein can be used and positioned in a variety of locations within a body. While specific examples may be provided herein, it is understood that that the working portions can be positioned in different regions of a body than those specifically described herein.

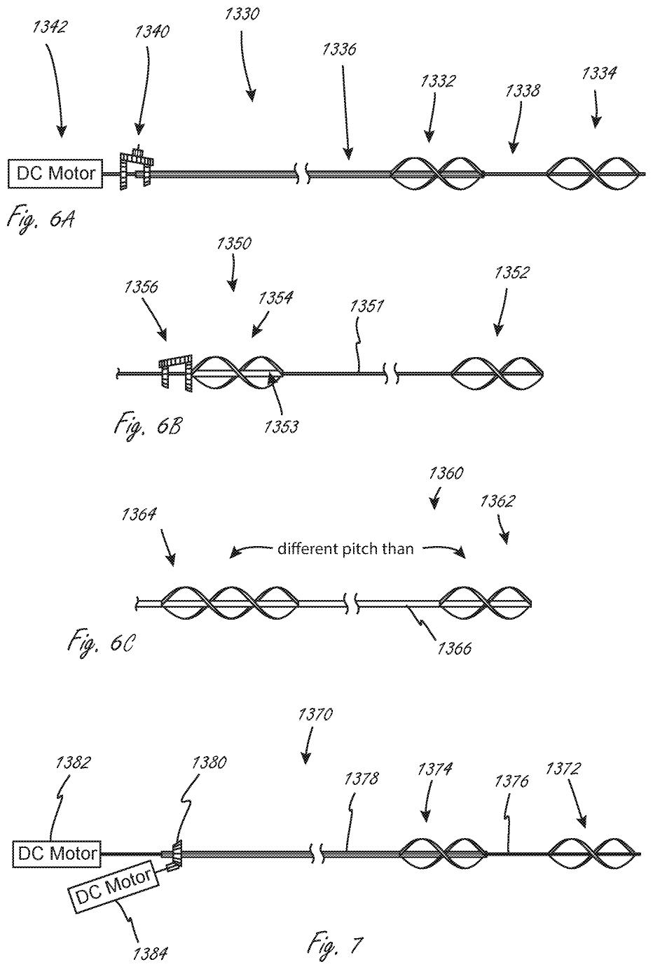

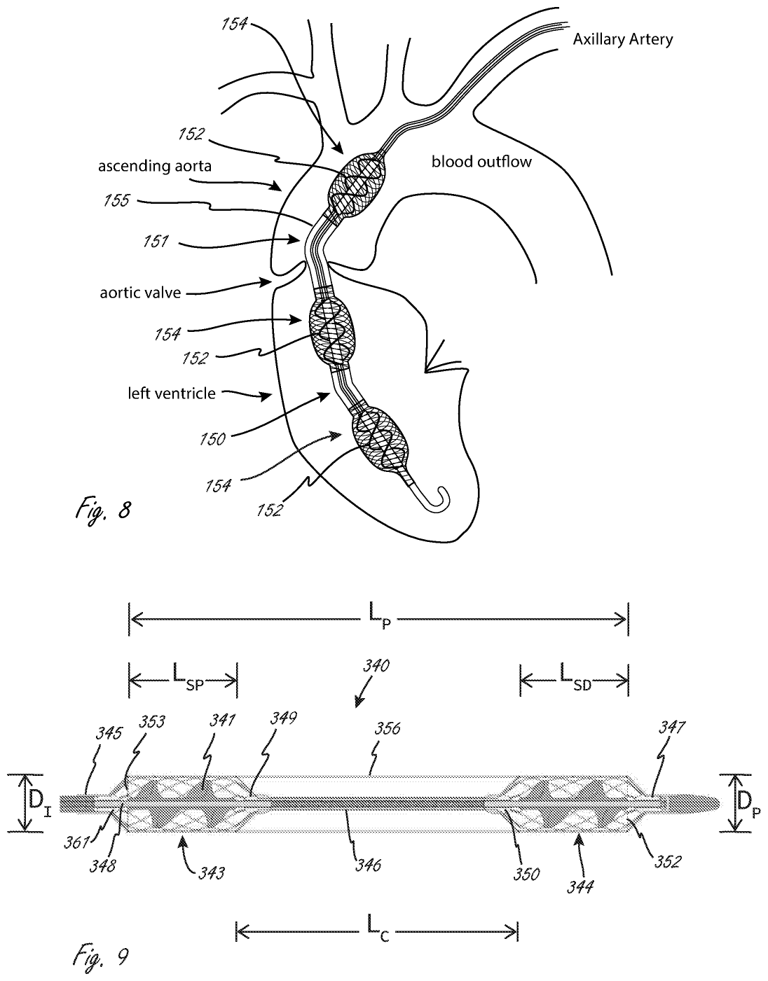

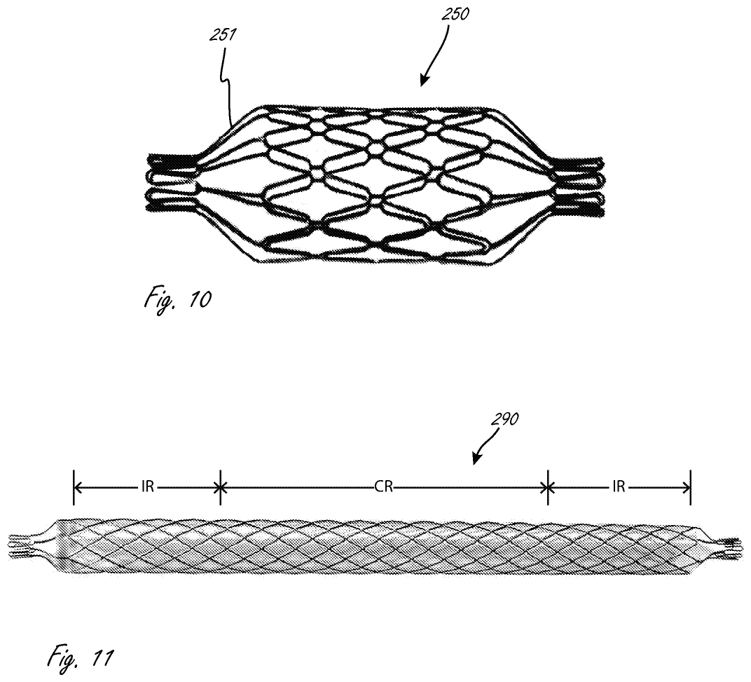

In any of the embodiments herein in which the catheter blood pump includes a plurality of impellers, the device can be adapted such that the impellers rotate at different speeds. FIG. 6A illustrates a medical device that includes gearset 1340 coupled to both inner drive member 1338 and outer drive member 1336, which are in operable communication with distal impeller 1334 and proximal impeller 1332, respectively. The device also includes motor 1342, which drives the rotation of inner drive member 1338. Inner drive member 1338 extends through outer drive member 1336. Activation of the motor 1332 causes the two impellers to rotate at different speeds due to an underdrive or overdrive ratio. Gearset 1340 can be adapted to drive either the proximal or distal impeller faster than the other. Any of the devices herein can include any of the gearsets herein to drive the impellers at different speeds.

FIG. 6B illustrates a portion of an alternative embodiment of a dual impeller device (1350) that is also adapted such that the different impellers rotate at different speeds. Gearset 1356 is coupled to both inner drive member 1351 and outer drive member 1353, which are coupled to distal impeller 1352 and proximal impeller 1354, respectively. The device also includes a motor like in FIG. 6A. FIGS. 6A and 6B illustrate how a gearset can be adapted to drive the proximal impeller slower or faster than the distal impeller.

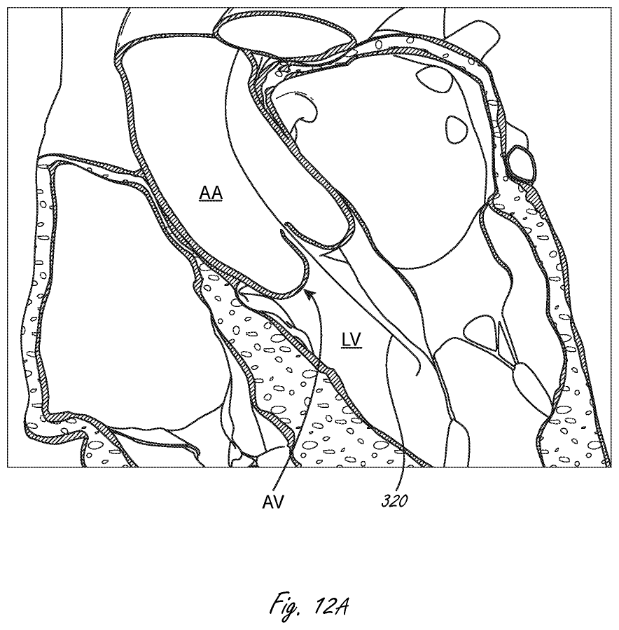

FIG. 7 illustrates an exemplary alternative embodiment of fluid pump 1370 that can rotate first and second impellers at different speeds. First motor 1382 drives cable 1376, which is coupled to distal impeller 1372, while second motor 1384 drives outer drive member 1378 (via gearset 1380), which is coupled to proximal impeller 1374. Drive cable 1376 extends through outer drive member 1378. The motors can be individually controlled and operated, and thus the speeds of the two impellers can be controlled separately. This system setup can be used with any system herein that includes a plurality of impellers.

In some embodiments, a common drive mechanism (e.g., cable and/or shaft) can drive the rotation of two (or more) impellers, but the blade pitch of the two impellers (angle of rotational curvature) can be different, with the distal or proximal impeller having a steeper or more gradual angle than the other impeller. This can produce a similar effect to having a gearset. FIG. 6C shows a portion of a medical device (1360) that includes common drive cable 1366 coupled to proximal impeller 1364 and distal impeller 1362, and to a motor not shown. The proximal impellers herein can have a greater or less pitch than the distal impellers herein. Any of the working portions (or distal portions) herein with a plurality of impellers can be modified to include first and second impellers with different pitches.

In any of the embodiments herein, the pump portion may have a compliant or semi-compliant (referred to generally together as “compliant”) exterior structure. In various embodiments, the compliant portion is pliable. In various embodiments, the compliant portion deforms only partially under pressure. For example, the central portion of the pump may be formed of a compliant exterior structure such that it deforms in response to forces of the valve. In this manner the exterior forces of the pump on the valve leaflets are reduced. This can help prevent damage to the valve at the location where it spans the valve.

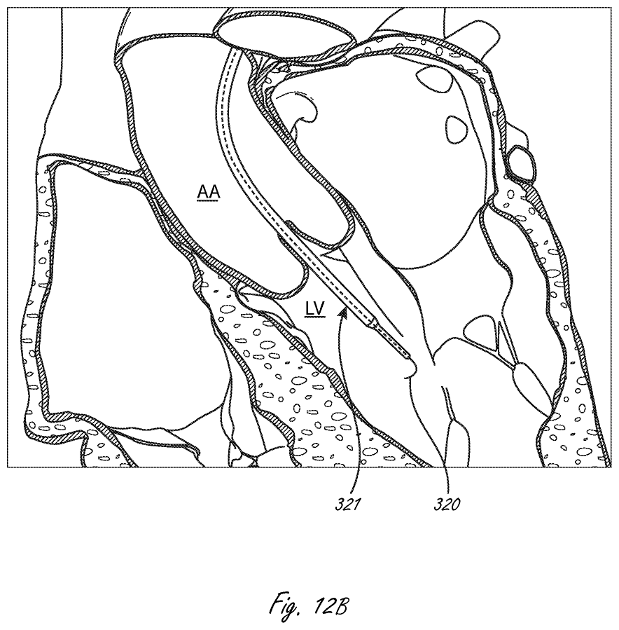

FIG. 8 illustrates an exemplary embodiment of a pump portion that includes first, second and third axially spaced impellers 152, each of which is disposed within an expandable member 154. Conduit 155 can extend along the length of the pump portion, as in described in various embodiments herein, which can help create and define the fluid lumen. In alternative embodiments, however, the first, second, and third impellers may be disposed within a single expandable member, similar to that shown in FIG. 1. In FIG. 8, a fluid lumen extends from a distal end to a proximal end, features of which are described elsewhere herein. The embodiment in FIG. 8 can include any other suitable feature, including methods of use, described herein.

The embodiment in FIG. 8 is also an example of an outer housing having at least one bend formed therein between a proximal impeller distal end and a distal impeller proximal end, such that a distal region of the housing distal to the bend is not axially aligned with a proximal region of the housing proximal to the bend along an axis. In this embodiment there are two bends 150 and 151 formed in the housing, each one between two adjacent impellers.

In a method of use, a bend formed in a housing can be positioned to span a valve, such as the aortic valve shown in FIG. 8. In this method of placement, a central impeller and distal-most impeller are positioned in the left ventricle, and a proximal-most impeller is positioned in the ascending aorta. Bend 151 is positioned just downstream to the aortic valve.

A bend such as bend 150 or 151 can be incorporated into any of the embodiments or designs herein. The bend may be a preformed angle or may be adjustable in situ.

In any of the embodiments herein, unless indicated to the contrary, the outer housing can have a substantially uniform diameter along its length.

In FIG. 8, the pump is positioned via the axillary artery, which is an exemplary method of accessing the aortic valve, and which allows the patient to walk and be active with less interruption. Any of the devices herein can be positioned via the axillary artery. It will be appreciated from the description herein, however, that the pump may be introduced and tracked into position in various manners including a femoral approach over the aortic arch.

One aspect of the disclosure is a catheter blood pump that includes a distal impeller axially spaced from a proximal impeller. Distal and proximal impellers may be axially spaced from each other. For example, the distal and proximal impellers may be connected solely by their individual attachment to a common drive mechanism. This is different from a single impeller having multiple blade rows or sections. A distal impeller as that phrase is used herein does not necessarily mean a distal-most impeller of the pump, but can refer generally to an impeller that is positioned further distally than a proximal impeller, even if there is an additional impeller than is disposed further distally than the distal impeller. Similarly, a proximal impeller as that phrase is used herein does not necessarily mean a proximal-most impeller of the pump, but can refer generally to an impeller that is positioned further proximally than a proximal impeller, even if there is an additional impeller than is disposed further proximally than the proximal impeller. Axial spacing (or some derivative thereof) refers to spacing along the length of a pump portion, such as along a longitudinal axis of the pump portion, even if there is a bend in the pump portion. In various embodiments, each of the proximal and distal impellers are positioned within respective housings and configured to maintain a precise, consistent tip gap, and the span between the impellers has a relatively more flexible (or completely flexible) fluid lumen. For example, each of the impellers may be positioned within a respective housing having relatively rigid outer wall to resist radial collapse. The sections between the impellers may be relatively rigid, in some embodiments the section is held open primarily by the fluid pressure within.

Although not required for the embodiments therein, there may be advantages to having a minimum axial spacing between a proximal impeller and a distal impeller. For example, a pump portion may be delivered to a target location through parts of the anatomy that have relatively tight bends, such as, for example, an aorta, and down into the aortic valve. For example, a pump portion may be delivered through a femoral artery access and to an aortic valve. It can be advantageous to have a system that is easier to bend so that it is easier to deliver the system through the bend(s) in the anatomy. Some designs where multiple impellers are quite close to each other may make the system, along the length that spans the multiple impellers, relatively stiff along that entire length that spans the multiple impellers. Spacing the impellers apart axially, and optionally providing a relatively flexible region in between the impellers, can create a part of the system that is more flexible, is easier to bend, and can be advanced through the bends more easily and more safely. An additional exemplary advantage is that the axial spacing can allow for a relatively more compliant region between the impellers, which can be positioned at, for example, the location of a valve (e.g., an aortic valve). Furthermore, there are other potential advantages and functional differences between the various embodiments herein and typical multistage pumps. A typical multistage pump includes rows of blades (sometimes referred to as impellers) in close functional spacing such that the rows of blades act together as a synchronized stage. One will appreciate that the flow may separate as it passes through the distal impeller. In various embodiments as described herein, distal and proximal impellers can be spaced sufficiently apart such that the flow separation from the distal impeller is substantially reduced (i.e., increased flow reattachment) and the localized turbulent flow is dissipated before the flow enters the proximal impeller.

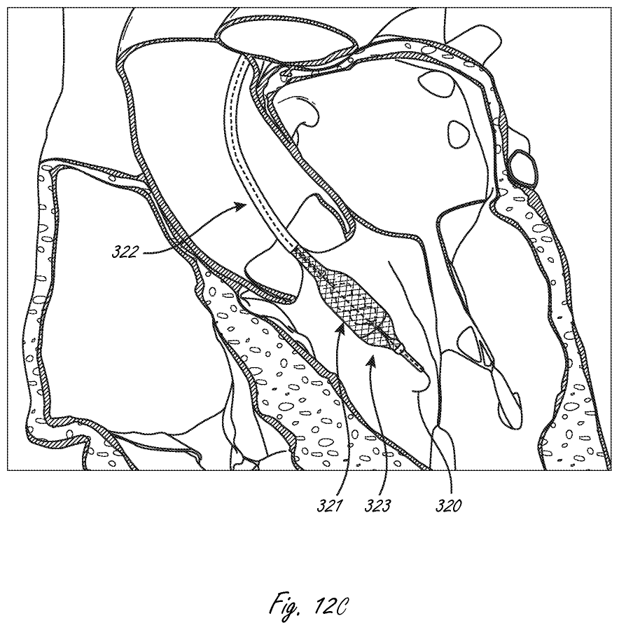

In any of the embodiments or in any part of the description herein that include a distal impeller and a proximal impeller, the axial spacing between a distal end of the proximal impeller and a proximal end of the distal impeller can be from 1.5 cm to 25 cm (inclusive) along a longitudinal axis of the pump portion, or along a longitudinal axis of a housing portion that includes a fluid lumen. The distance may be measured when the pump portion, including any impellers, is in an expanded configuration. This exemplary range can provide the exemplary flexibility benefits described herein as the pump portion is delivered through curved portions of the anatomy, such as, for example, an aortic valve via an aorta. FIG. 9 (shown outside a patient in an expanded configuration) illustrates length Lc, which illustrates an axial spacing between impellers, and in some embodiments may be from 1.5 cm to 25 cm as set forth herein. In embodiments in which there may be more than two impellers, any two adjacent impellers (i.e., impellers that do not have any other rotating impeller in between them) may be spaced axially by any of the axial spacing distances described herein.

While some embodiments include a proximal impeller distal end that is axially spaced 1.5 cm to 25 cm from a distal impeller proximal end along an axis, the disclosure herein also includes any axial spacings that are subranges within that general range of 1.5 cm to 25 cm. That is, the disclosure includes all ranges that have any lower limit from 1.5 and above in that range, and all subranges that have any upper limit from 25 cm and below. The examples below provide exemplary subranges. In some embodiments, a proximal impeller distal end is axially spaced 1.5 cm to 20 cm from a distal impeller proximal end along an axis, 1.5 cm to 15 cm, 1.5 cm to 10 cm, 1.5 cm to 7.5 cm, 1.5 cm to 6 cm, 1.5 cm to 4.5 cm, 1.5 cm to 3 cm. In some embodiments the axial spacing is 2 cm to 20 cm, 2 cm to 15 cm, 2 cm to 12 cm, 2 cm to 10 cm, 2 cm to 7.5 cm, 2 cm to 6 cm, 2 cm to 4.5 cm, 2 cm to 3 cm. In some embodiments the axial spacing is 2.5 cm to 15 cm, 2.5 cm to 12.5 cm, 2.5 cm to 10 cm, 2.5 cm to 7.5 cm, or 2.5 cm to 5 cm (e.g., 3 cm). In some embodiments the axial spacing is 3 cm to 20 cm, 3 cm to 15 cm, 3 cm to 10 cm, 3 cm to 7.5 cm, 3 cm to 6 cm, or 3 cm to 4.5 cm. In some embodiments the axial spacing is 4 cm to 20 cm, 4 cm to 15 cm, 4 cm to 10 cm, 4 cm to 7.5 cm, 4 cm to 6 cm, or 4 cm to 4.5 cm. In some embodiments the axial spacing is 5 cm to 20 cm, 5 cm to 15 cm, 5 cm to 10 cm, 5 cm to 7.5 cm, or 5 cm to 6 cm. In some embodiments the axial spacing is 6 cm to 20 cm, 6 cm to 15 cm, 6 cm to 10 cm, or 6 cm to 7.5 cm. In some embodiments the axial spacing is 7 cm to 20 cm, 7 cm to 15 cm, or 7 cm to 10 cm. In some embodiments the axial spacing is 8 cm to 20 cm, 8 cm to 15 cm, or 8 cm to 10 cm. In some embodiments the axial spacing is 9 cm to 20 cm, 9 cm to 15 cm, or 9 cm to 10 cm. In various embodiments, the fluid lumen between the impellers is relatively unsupported.

In any of the embodiments herein the one or more impellers may have a length, as measured axially between an impeller distal end and an impeller proximal end (shown as “L_{SD}” and “L_{SP}”, respectively, in FIG. 9), from 0.5 cm to 10 cm, or any subrange thereof. The examples below provide exemplary subranges. In some embodiments the impeller axial length is from 0.5 cm to 7.5 cm, from 0.5 cm to 5 cm, from 0.5 cm to 4 cm, from 0.5 cm to 3 cm, from 0.5 cm to 2, or from 0.5 cm to 1.5 cm. In some embodiments the impeller axial length is from 0.8 cm to 7.5 cm, from 0.8 cm to 5 cm, from 0.8 cm to 4 cm, from 0.8 cm to 3 cm, from 0.8 cm to 2 cm, or from 0.8 cm to 1.5 cm. In some embodiments the impeller axial length is from 1 cm to 7.5 cm, from 1 cm to 5 cm, from 1 cm to 4 cm, from 1 cm to 3 cm, from 1 cm to 2 cm, or from 1 cm to 1.5 cm. In some embodiments the impeller axial length is from 1.2 cm to 7.5 cm, from 1.2 cm to 5 cm, from 1.2 cm to 4 cm, from 1.2 cm to 3 cm, from 1.2 to 2 cm, or from 1.2 cm to 1.5 cm. In some embodiments the impeller axial length is from 1.5 cm to 7.5 cm, from 1.5 cm to 5 cm, from 1.5 cm to 4 cm, from 1.5 cm to 3 cm, or from 1.5 cm to 2 cm. In some embodiments the impeller axial length is from 2 cm to 7.5 cm, from 2 cm to 5 cm, from 2 cm to 4 cm, or from 2 cm to 3 cm. In some embodiments the impeller axial length is from 3 cm to 7.5 cm, from 3 cm to 5 cm, or from 3 cm to 4 cm. In some embodiments the impeller axial length is from 4 cm to 7.5 cm, or from 4 cm to 5 cm.

In any of the embodiments herein the fluid lumen can have a length from a distal end to a proximal end, shown as length Lp in FIG. 9. In some embodiments the fluid lumen length Lp is from 4 cm to 40 cm, or any subrange therein. For example, in some embodiments the length Lp can be from 4 cm to 30 cm, from 4 cm to 20 cm, from 4 cm to 18 cm, from 4 cm to 16 cm, from 4 cm to 14 cm, from 4 cm to 12 cm, from 4 cm to 10 cm, from 4 cm to 8 cm, from 4 cm to 6 cm.

In any of the embodiments herein the housing can have a deployed diameter, at least the location of an impeller (and optionally at a location between impellers), shown as dimension Dp in FIG. 9. In some embodiments Dp can be from 0.3 cm to 1.5 cm, or any subrange therein. For example, Dp may be from 0.4 cm to 1.4 cm, from 0.4 cm to 1.2 cm, from 0.4 cm to 1.0 cm, from 0.4 cm to 0.8 cm, or from 0.4 cm to 0.6 cm. In some embodiments, Dp may be from 0.5 cm to 1.4 cm, from 0.5 cm to 1.2 cm, from 0.5 cm to 1.0 cm, from 0.5 cm to 0.8 cm, or from 0.5 cm to 0.6 cm. In some embodiments Dp may be from 0.6 cm to 1.4 cm, from 0.6 cm to 1.2 cm, from 0.6 cm to 1.0 cm, or from 0.6 cm to 0.8 cm. In some embodiments Dp may be from 0.7 cm to 1.4 cm, from 0.7 cm to 1.2 cm, from 0.7 cm to 1.0 cm, or from 0.7 cm to 0.8 cm.

In any of the embodiments herein an impeller can have a deployed diameter, shown as dimension Di in FIG. 9. In some embodiments Di can be from 1 mm-30 mm, or any subrange therein. For example, in some embodiments Di may be from 1 mm-15 mm, from 2 mm-12 mm, from 2.5 mm-10 mm, or 3 mm-8 mm.

In any of the embodiments herein, a tip gap exists between an impeller outer diameter and a fluid lumen inner diameter. In some embodiments the tip gap can be from 0.01 mm-1 mm, such as 0.05 mm to 0.8 mm, or such as 0.1 mm-0.5 mm.

In any of the embodiments herein that includes multiple impellers, the axial spacing between impellers (along the length of the pump portion, even if there is a bend in the pump portion) can be from 2 mm to 100 mm, or any combination of upper and lower limits inclusive of 5 and 100 mm (e.g., from 10 mm-80 mm, from 15 mm-70 mm, from 20 mm-50 mm, 2 mm-45 mm, etc.).

Any of the pump portions herein that include a plurality of impellers may also include more than two impellers, such as three, four, or five impellers (for example).

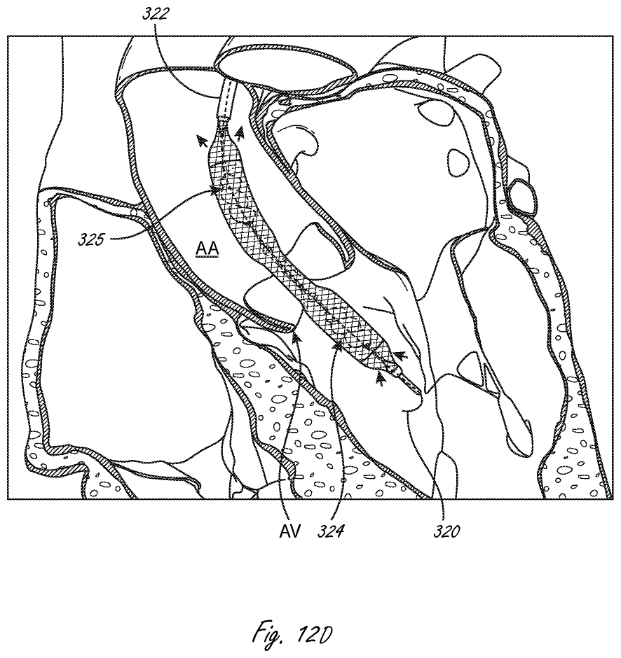

FIG. 10 illustrates an expandable scaffold 250 that may be one of at least two expandable scaffolds of a pump portion, such as the expandable scaffolds in FIGS. 3A-3D, wherein each expandable scaffold at least partially surrounds an impeller. The scaffold design in FIG. 10 has proximal struts 251 (only one labeled) extending axially therefrom. Having a separate expandable scaffold 250 for each impeller provides for the ability to have different geometries for any of the individual impellers. Additionally, this design reduces the amount of scaffold material (e.g., Nitinol) over the length of the expandable blood conduit, which may offer increased tracking when sheathed. A potential challenge with these designs may include creating a continuous membrane between the expandable scaffolds in the absence of an axially extending scaffolding material (see FIG. 3A). Any other aspect of the expandable scaffolds or members herein, such as those described in FIGS. 3A-3D, may be incorporated by reference into this exemplary design. Struts 251 may be disposed at a pump inflow or outflow. Struts 251 may be proximal struts or they may be distal struts.

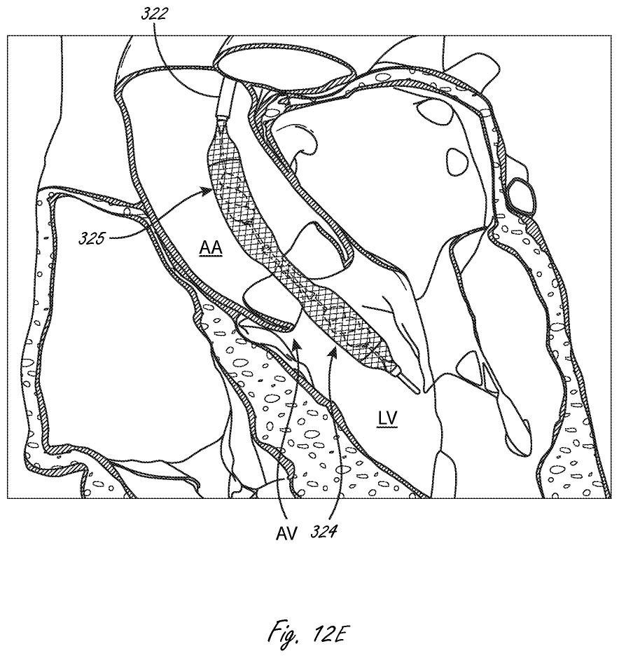

FIG. 11 show an exemplary scaffold along an length of the blood conduit. Central region “CR” may be axially between proximal and distal impellers. Central region “CR” flexibility is increased relative to scaffold impeller regions “IR” due to breaks or discontinuities in the scaffold pattern in the central region. The scaffold has relatively more rigid impeller sections “IR” adjacent the central region where impellers may be disposed (not shown). The relatively increased rigidity in the impeller regions IR may help maintain tip gap and impeller concentricity. This pump scaffold pattern provides for a flexibility distribution, along its length, of a proximal section of relatively less flexibility (“IR”), a central region “CR” of relatively higher flexibility, and a distal section “IR” of relatively less flexibility than the central region. The relatively less flexible sections (i.e., the two IR regions) are where proximal and distal impellers may be disposed (not shown but other embodiments are fully incorporated herein in this regard), with a relatively more flexible region in between. Exemplary benefits of the relative flexibility in these respective sections are described elsewhere herein. FIG. 11 is an example of a scaffold that is continuous from a first end region to a second end region, even though there are breaks or discontinuities in some locations of the scaffold. There is at least one line that can be traced along a continuous structural path from a first end region to a second end region.

The following disclosure provides exemplary method steps that may be performed when using any of the blood pumps, or portions thereof, described herein. It is understood that not all of the steps need to be performed, but rather the steps are intended to be an illustrative procedure. It is also intended that, if suitable, in some instances the order of one or more steps may be different. Before use, the blood pump can be prepared for use by priming the lumens (including any annular spaces) and pump assembly with sterile solution (e.g., heparinized saline) to remove any air bubbles from any fluid lines. The catheter, including any number of purge lines, may then be connected to a console. Alternatively, the catheter may be connected to a console and/or a separate pump that are used to prime the catheter to remove air bubbles.

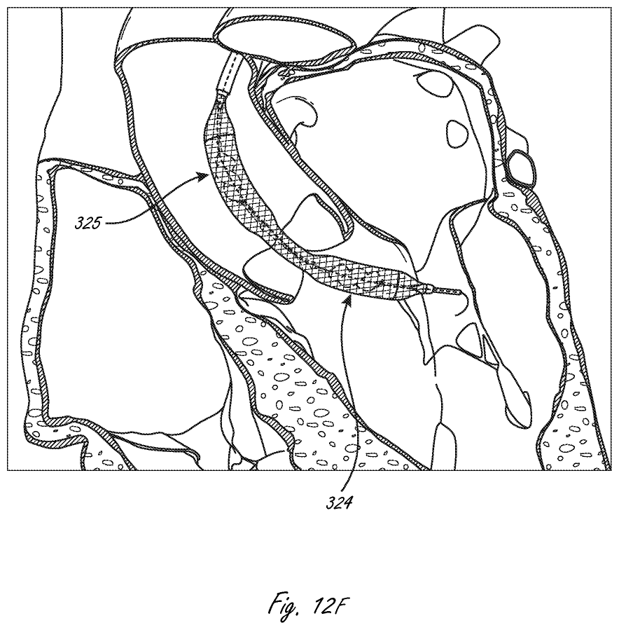

After priming the catheter, access to the patient's vasculature can be obtained (e.g., without limitation, via femoral access) using an appropriately sized introducer sheath. Using standard valve crossing techniques, a diagnostic pigtail catheter may then be advanced over a, for example, 0.035″ guide wire until the pigtail catheter is positioned securely in the target location (e.g., left ventricle). The guidewire can then be removed and a second wire 320 (e.g., a 0.018″ wire) can be inserted through the pigtail catheter. The pigtail catheter can then be removed (see FIG. 12A), and the blood pump 321 (including a catheter, catheter sheath, and pump portion within the sheath; see FIG. 12B) can be advanced over the second wire towards a target location, such as spanning an aortic valve “AV,” and into a target location (e.g., left ventricle “LV”), using, for example, one or more radiopaque markers to position the blood pump.

Once proper placement is confirmed, the catheter sheath 322 (see FIG. 12C) can be retracted, exposing first a distal region of the pump portion. In FIG. 12C a distal region of an expandable housing has been released from sheath 322 and is expanded, as is distal impeller 324. A proximal end of housing 323 and a proximal impeller are not yet released from sheath 322. Continued retraction of sheath 322 beyond the proximal end of housing 323 allows the housing 323 and proximal impeller 325 to expand (see FIG. 12D). The inflow region (shown with arrows even though the impellers are not yet rotating) and the distal impeller are in the left ventricle. The outflow (shown with arrows even though the impellers are not rotating yet) and proximal impeller are in the ascending aorta AA. The region of the outer housing in between the two impellers, which may be more flexible than the housing regions surrounding the impellers, as described in more detail herein, spans the aortic valve AV. In an exemplary operating position as shown, an inlet portion of the pump portion will be distal to the aortic valve, in the left ventricle, and an outlet of the pump portion will be proximal to the aortic valve, in the ascending aorta (“AA”).

The second wire (e.g., an 0.018″ guidewire) may then be moved prior to operation of the pump assembly (see FIG. 12E). If desired or needed, the pump portion can be deflected (active or passively) at one or more locations as described herein, as illustrated in FIG. 12F. For example, a region between two impellers can be deflected by tensioning a tensioning member that extends to a location between two impellers. The deflection may be desired or needed to accommodate the specific anatomy. As needed, the pump portion can be repositioned to achieve the intended placement, such as, for example, having a first impeller on one side of a heart valve and a second impeller on a second side of the heart valve. It is understood that in FIG. 12F, the pump portion is not in any way interfering or interacting with the mitral valve, even if it may appear that way from the figure.

For some catheter blood pumps, it may be desirable to have collapsible impellers to, for example, reduce the delivery profile of the impeller and pump portion as much as possible for delivery and placement. Once the one or more collapsible impellers are expanded and in use, however, the impeller(s) must be able to provide a desired flow rate, and it is generally desirable to reduce the impeller(s) rotation rate (RPM) as much as possible to minimize the amount of wear on system components, such as one or more bearings. Optimizing impeller performance while at the same time providing impeller(s) that can be reliably collapsed and expanded can thus be an important aspect of pump design for some catheter blood pumps with one or more collapsible impellers. Systems that include more than one impeller may be able to achieve the desired flow characteristics by utilizing more than one impeller. Advantageously, each of the impellers can perform less work than would be required by a single impeller pump. Because the work of the pump can be divided amongst more than one impeller, each impeller can be smaller than might be required by a single impeller pump. The individual impellers may thus be designed to have one or more of a smaller expanded profile, less stiff material, or more reliable collapsibility. By having these and/or other similar characteristics, it may be easier to design individual impellers of a multi-impeller blood pump because each individual pump can be smaller and more flexible than an impeller of a single impeller pump. An impeller in a single impeller pump may need to be more rigid and larger to perform all of the work of the pump, which may make it more difficult to collapse to a desired lower delivery profile. Multi-impeller pumps may thus enable the blood pump to have a smaller French size.

It may be desirable for one or more impellers in a pump portion of a catheter blood pump to be configured to be an at least partial radial flow (centrifugal) impeller, or at least not solely axial flow. At least partial radial flow refers to the impeller generating flow that has some radial component, and includes mixed impellers that create both axial flow and radial flow. Impellers that are configured to generate some radial flow may increase flow performance and/or allow for a reduction in pump speed, which can be beneficial to the system. Impellers that are configured to generate some radial flow may increase performance, which may allow the number of impeller blades to be reduced, which may simplify the manufacturing of the impeller, such as when an impeller is molded.

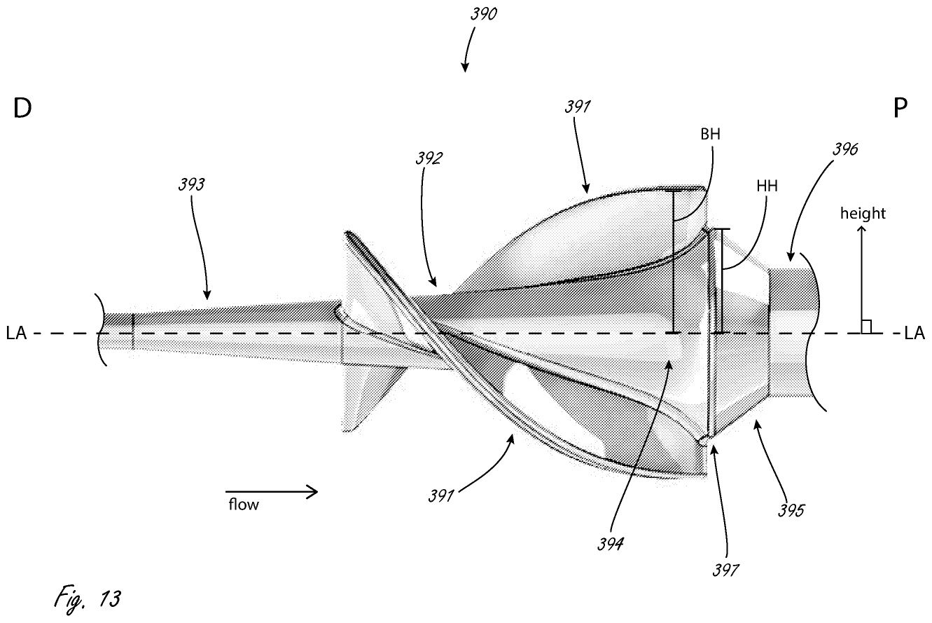

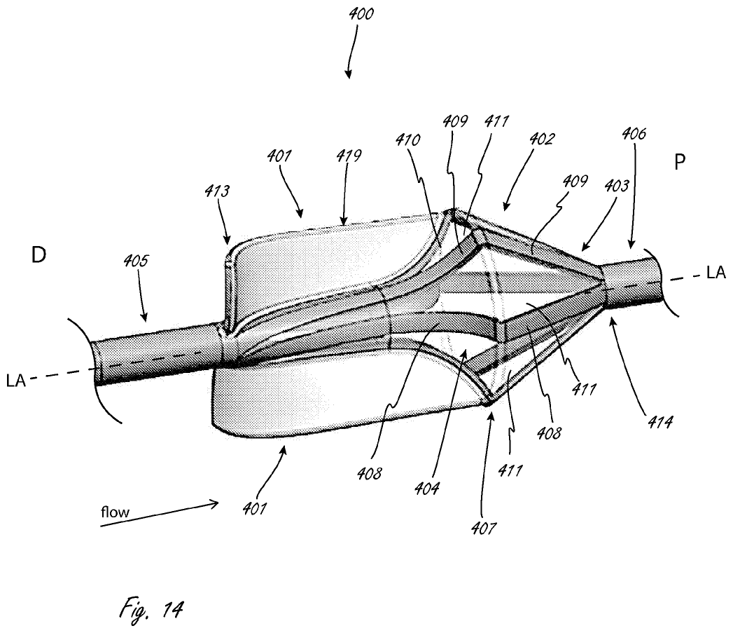

FIGS. 13 and 14 illustrate exemplary collapsible impellers that are configured to create at least some amount of radial flow when rotated during use. The impellers in FIGS. 13 and 14 may be incorporated with any other aspect of any other embodiments herein, including pumps that include one or more impellers. For example, pump portions herein may include one or more impellers shown in FIGS. 13 and 14, and may also include any suitable expandable housing herein. FIG. 13 shows exemplary impeller 390, which includes hub 392 and blades 391 extending radially outward from hub 392. Hub 392 includes crest 397, which refers generally to a region of the hub with a greatest outermost radial dimension measured orthogonally from a longitudinal axis LA (the dimension also referred to herein as “height,” as labeled in FIG. 13, which may also be referred to herein as a radius). The height of the hub decreases in both axial directions away from crest 397, although the crest region may be axially longer than is shown in FIG. 13. That is, the crest region may extend along a greater axial length of the hub than is shown in FIG. 13, in which case the crest may have more of a cylindrical configuration. In this exemplary embodiment, the hub decreases in height in region 395 proximal to the crest, and decreases in height distal to crest in regions 394 and 393. In any alternative embodiment, region 393 distal to the blades may have a substantially constant height or radius (i.e., the height may not decrease, as is shown in FIG. 13), while region 394 in the bladed region has a varying height or radius. The combination of the ramped hub configuration and blade configuration imparts some radial flow to the fluid as impeller 390 rotates. This may provide the performance improvements noted herein. The proximally tapered region 395 is configured to entrain blood flow.

Impeller 390, including at least a portion of hub 392, may be adapted to be collapsed within a delivery device such as a sheath, which is described elsewhere herein. For example, the impeller may be made from one or more deformable polymeric materials, and may be molded. Exemplary concepts related to collapsible impellers may be described in U.S. Pat. No. 7,841,976, which is fully incorporated by reference herein for all purposes.

The impeller in FIG. 13 is an example of an impeller that includes at least one blade and an expandable hub (e.g., hub 392, including portion 395), wherein the expandable hub is adapted to be expanded from a delivery configuration towards a fully deployed configuration (e.g., as shown in FIG. 13), the fully deployed configuration having at least one region that is greater in outer dimension relative to the delivery configuration.

The impeller in FIG. 13 is also an example of an expandable impeller comprising at least one blade and a hub, the hub having a proximal portion in which the hub outer surface tapers radially inward in a proximal direction (e.g., in region 395).

The impeller in FIG. 13 is also an example of a collapsible impeller, the collapsible impeller including at least one deformable blade (e.g., blades 391) that is adapted to at least partially fold/wrap around a long impeller axis when collapsed, the impeller further comprising a collapsible structure (e.g., hub 394) different than the at least one deformable blade, the collapsible structure having at least one surface that is configured to be radially collapsed toward the long axis when collapsed within the radially outer shaft.

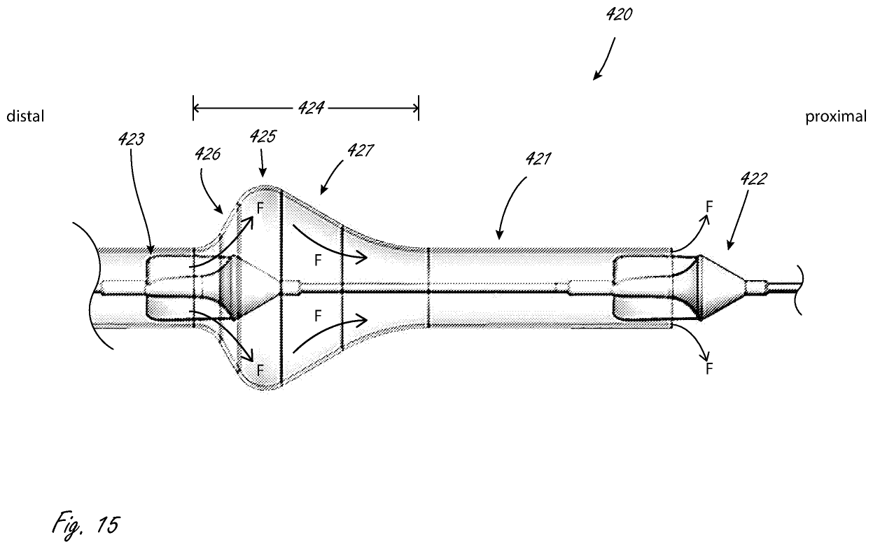

The impeller 390 may be a proximal impeller and/or a distal impeller of a blood pump. It may be part of a single impeller or a multi-impeller blood pump. In some embodiments a proximal region of impeller 390 extends proximally out of any of the collapsible blood conduits herein, such as is shown in exemplary FIG. 15.

An important consideration for designing some collapsible impellers is making sure the collapsible impeller can be collapsed reliably, and when expanded and in use it has the necessary strength to provide the desired flow characteristics. For example, it may be easy to make a very flexible impeller that is easy to collapse, but when the impeller is expanded and rotated at the necessary RPMs, it may deform easily and/or fail to provide the necessary flow rates due to a lack of strength/stiffness. Some uses of collapsible impellers thus present an additional design consideration compared to non-collapsible impellers that are made of much stiffer material, such as metal. Any collapsible impellers herein may be made of one or more polymeric materials.

Impeller 390 includes first and second blades, both of which have front and back surfaces that are orthogonal to long axis LA, as shown in FIG. 13.

FIG. 14 illustrates an exemplary collapsible impeller, at least a portion of which is adapted to be pressurized and/or inflated with a fluid to increase the stiffness of at least a portion of the impeller. The exemplary impeller in FIG. 14 is in some ways similar to the impeller in FIG. 13 (e.g., a similar general outer profile of the impeller in expanded configurations), but is different in other ways. One or more aspects of impeller 400 in FIG. 14 may facilitate collapse and/or help reduce the sheathing force needed to collapse the impeller. Impeller 400 is also adapted such that the stiffness of at least one portion of the impeller (e.g., the hub) may be increased when fluid is advanced through and/or within the impeller hub. The hub may be in fluid communication with a fluid source (e.g., a purge fluid source disposed outside of the patient, or a separate inflation fluid source), and when the fluid is advanced (e.g. pumped) through and/or into the hub, the fluid pressure can increase at the hub surface and thereby increase the stiffness of the hub. Increasing the stiffness of the impeller with fluid may allow the impeller to be designed to be more flexible and easier to deform for collapse, while allowing the impeller to have the necessary/desired strength and rigidity when in use. The hub of impeller 390 in FIG. 13 may also be adapted (e.g., material and/or thickness) such that an increase in fluid pressure increases the stiffness of the hub, which may allow it to be designed to be more easily deformable for collapse.

Impeller 400 is configured to be collapsed for delivery inside a device (e.g., a delivery sheath) that maintains it in a collapsed configuration. When impeller 400 is deployed from the delivery sheath, at least a portion of impeller 400 may self-expand to a self-deployed configuration. Self-expansion of the hub may be caused at least partially due to the at-rest self-deployed configuration of one or more support members 408 and 409, two of which are labeled in FIG. 14. In some embodiments the support members 408 and 409 (e.g., support arms) may comprise a material that is adapted to be collapsed and passively return to an at-rest expanded configuration when deployed (e.g., nitinol, or other similar resilient material). Self-expansion of the hub may also or alternatively be caused at least partially by the molded shape of the hub.

Any self-expanded impeller configuration herein may be the same or substantially the same configuration as a final deployed configuration ready for use (i.e., ready for rotation). In some instances the self-deployed configurations may not be the same or substantially the same as a final deployed configuration, and another step may be required to transition the impeller to a final deployed configuration. For example, advancing fluid through and/or into the impeller may transition the impeller to final deployed configuration. Substantially the same configuration in this context refers to a configuration that is essentially the same, even if there is some minor change in configuration that occurs after a subsequent step (e.g., fluid delivery, purge fluid delivery, etc.). For example, advancing fluid into the impeller may cause the outer profile of the hub to change only slightly due to an increase in pressure, but not significantly change.

An impeller hub may also include one or more regions that are relatively less stiff than the support members, such as support members 408 and 409. Hub 402 includes sections or regions 411 disposed in between the support members, which may be less stiff than support arms 408, 409, 410, 410′). Hub sections or regions 411 may be relatively thin flexible material (e.g., a polymeric material such as rubber). The region(s) 411 of hub are adapted such that fluid delivery through/within the hub, which increases the fluid pressure on the hub, increases the stiffness of the hub. This can increase the strength of the impeller when in use, the advantages of which are discussed herein. In some embodiments the regions 411 can be a continuous, uninterrupted material, to which the support members (e.g., 408, 409, 410) are secured (directly or indirectly). In some embodiments the regions 411 may be separate regions that do not form a continuous region of material, and can be secured to one or more support members. In some embodiments, the support members may be formed of the same material as regions 411, and may be thicker than regions 411 to provide enhanced stiffness compared to regions 411.

In this exemplary embodiment the blades 401 (two in this embodiment) extend radially outward from the hub, with one or more support members disposed circumferentially in between the blades. As an example only, in some embodiments the support members (e.g., 408, 409, etc.) and hub regions 405 and 406 are made from the same starting material, that is, they are unitary. Removing material in a central region of the starting tubular member can thereby form and define the support members. The support members may then be formed to have any desired configuration towards which they will naturally revert upon the removal of a collapse force. As an example only, in some embodiments the blades and hub section 411 are molded together as a unit. The more flexible hub section 611 and blades may then be secured to the stiffer member that includes hub section 405/406 and support members 408, 409, etc.

Support arms 408 and 409 extend along the hub in bladed region 404 and proximal region 403, including crest region 407. In some embodiments, the support members are disposed and coupled external to hub region(s) 411. In some embodiments, the support members are disposed and coupled radially inside hub region(s) 411.

Impellers 390 and 400 may include more than two blades. For example, impeller 400 may have a third impeller blade disposed between support member 408 and 409. Any of the blades on a given impeller may have substantially the same configuration.

Impeller 400 may include more or fewer numbers of support members. For example, in alternative embodiments, impeller 400 may have a single support member in between circumferentially adjacent blades. Any suitable impeller herein may have from two to twenty support members, or more (for example, if the support members are made relatively thin).

In alternative embodiments, the greatest blade height “BH” dimension (see FIG. 13) of blades 401 is greater than the greatest hub height “HH” (see FIG. 13). Impeller 400 can be described as having similar sections, regions, or portions to impeller 390 in FIG. 13. For example, impeller 400 has a crest 407, at which the hub height is greatest (“HH”). Impeller 400 includes a proximal region 403 whose height decreases in a the proximal direction towards hub 406. The configuration of proximal region 403 (as well as region 395 in FIG. 29) can help facilitate collapse of the impeller. Any other feature of impeller 390 may be incorporated with or into impeller 400.

In alternative embodiments, proximal impeller region 403 and proximal impeller region 395 may have configurations other than the conical configurations that are shown. For example, an outer surface of proximal region 403 may be concave (in a section taken through the long axis of the impeller). This is similar to the general configuration of the distal hub region 404 in impeller 400, which can be seen at the blade 401/hub interface region.

Impeller 400 is an example of an impeller that is configured to be pressurized and/or inflated with an inflation fluid, which if incorporated into a catheter blood pump, may be made to be in fluid communication with a fluid source (e.g., purge fluid or an inflation fluid) such that delivery of fluid from the fluid source increases pressure within the inflatable impeller and increases the stiffness of at least a portion of the impeller (e.g., the hub portion of the impeller).

In some embodiments, impeller 400 is a collapsible impeller that has a collapsed delivery configuration and a deployed configuration, wherein the impeller hub may have an internal volume that is greater in a fully deployed configuration than in a collapsed delivery configuration. The internal volume in impeller 400 may be considered a volume within the inner hub surfaces (which are exposed to fluid from a fluid/pressure source) and between blade distal end region 413 and proximal end region 414.

In this embodiment, impeller blades 401 are not inflatable, although they are collapsible, and at least a portion of one or more blades are configured and adapted to collapse down relative to long axis LA. In some embodiments, blades 401 can have a thickness that is greater than a thickness of material in hub region(s) 411.

In some embodiments, impeller 400 has an internal impeller volume that comprises an internal non-bladed region (e.g., region 403) volume and an internal bladed region (e.g., 404) volume. The internal non-bladed region volume may be greater than, less than, or the same as the internal bladed region volume. Non-bladed herein refers to axial regions that are void of blades, and bladed refers to axial regions that include blades.

In some embodiments, material in region(s) 411 of impeller 400 may be compliant or semi-compliant. During the pressurization/inflation process, the configuration of the region(s) 411 may change initially, but after a continued increase in fluid pressure, the configuration may not change substantially (i.e., there may be either no change or some minor degree of configuration change), and the hub becomes stiffer, advantages of which are described herein.

In some embodiments, fluid pressure within the hub may be used to a greater extent to expand the impeller to the fully deployed configuration. That is, the impeller may not self-expand to an almost fully deployed configuration, and fluid pressure causes a greater configuration change than in some embodiments in which the support members cause a self-expansion to a self-expanded configuration that is closer to the fully deployed configuration. For example only, impeller 400 may not, in some embodiments, include hub support members such as member 408 and 409. The inflatable and expandable hub can still be adapted (e.g., with a material and/or thickness) to be inflatable and deformable, but without support members does not self-deploy as close to the fully deployed configuration as it may in some other embodiments. Fluid expansion of the hub may thus contribute more to expansion towards the fully deployed configuration in these embodiments.

In embodiments that include one or support members (e.g., 408-410), the support members may have biased configurations (at rest) that do not extend as far radially outward as they are in the fully deployed configuration. In these embodiments, additional fluid pressure can cause them, or at least portions of them (e.g., the crest region) to expand further radially outward. By biasing one or more support members to a smaller at-rest radially height, it may be easier to collapse them than if they are biased to revert to a configuration in which their height is greater. Any of the suitable embodiments herein that include one or more support members can have support members that are biased to a configuration that has a smaller max height than a fully deployed configuration of the support members after fluid (i.e., liquid or gas) inflation.

In impeller embodiments that include proximal regions like proximal region 403, the proximal region may not decrease in height along the entire axial length of the proximal region, but may decrease in height only in a region. The proximal region may assume a wide variety of configurations, although there may be a benefit to the proximal end of the proximal region having a smaller height than the distal end of the proximal region (e.g., to make it easier to collapse).

Impellers 390 and 400 as shown have blade proximal ends that are adjacent to the hub crest region. The bladed region of the hubs in impellers 390 and 400 gradually increases in height between a distal end of the bladed region and a proximal end of the bladed region. In other embodiments there may not be a continuous increase in height. For example, there may be one or more discrete regions along which the height does not increase with the individual regions. The bladed region of the hubs include a trumpet shaped region, as shown, wherein the height gradually increases in a distal-to-proximal direction.

Impellers 390 and 400 are examples of impellers that have hubs with bladed regions (e.g., 404), and non-bladed regions (e.g., 403). In these examples the bladed and non-bladed regions have heights that vary along at least a portion of the region.

Impellers 390 and 400 are examples of impellers that have proximal non-bladed expandable regions that include one or more re-sheathing guides. Examples of the one or more re-sheathing guides include the outer configuration and one or more support members, such as support members 408 and 409.

Impellers 390 and 400 are examples of impellers including at least one blade and an and expandable and collapsible hub, the expandable hub adapted to be expanded from a collapsed delivery configuration towards a fully deployed configuration, the fully deployed configuration having at least one region that is greater in outer dimension relative to the region in the delivery configuration.

Impellers 390 and 400 are examples of expandable impellers that include at least one blade and a hub, the hub having a proximal portion in which the hub outer surface has a height that is greater at a distal end than at a proximal end.

Impellers 390 and 400 are examples of deformable impellers, the deformable impellers including an expandable portion and a second portion extending from the expandable portion, optionally wherein the second portion comprises at least one impeller blade, and optionally wherein the expandable portion includes at least part of an impeller hub. The expandable portion in each of impellers 390 and 400 includes a non-bladed region and a bladed-region.

Impellers 390 and 400 are examples of deformable impellers that include one or more blades that are adapted and configured to self-deploy when released from a delivery device (e.g., outer sheath). When collapsed, at least a portion of the blades wrap or curl relative to a long axis of the impeller. The blades may comprise a polymeric, flexible material. The proximal, non-bladed regions of the impellers 390 and 400 are configured to collapse radially inward towards the long axis, similar to how a traditional umbrella might collapse.

Either of impellers 390 and 400 may be proximal impellers, they may be distal impellers, or in pumps with multiple impellers, more than one of the impellers may be impeller 390 or 400, or any alternative thereof described herein.

Impellers 390 and 400 are examples of collapsible impellers, the collapsible impeller including at least one deformable blade that is adapted to at least partially collapse toward a long impeller axis when collapsed, the impeller further comprising a collapsible structure different than the at least one deformable blade, the collapsible structure having at least one surface that is configured to be radially collapsed toward the long axis when collapsed.

In some alternatives, the blades in impeller 400 are skinned, as that term is ordinarily understood, wherein the blades further includes one or more support elements to support the skinned blade. For example, the one or more support elements may be stiffer than the blade, such as one or more nitinol support elements disposed at and following the radially outer edge of the blade and secured to the blade. The support element may extend from hub 402, follow the outer edge of the blade, and may continue to at least the crest region 407, and optionally all the way to the proximal end 414. The one or more blades may comprise a thin polymeric material.

In some embodiments impeller 390 or 400 is disposed at or adjacent an outflow of the pump portion. The impeller may extend proximally beyond the proximal end of the blood conduit or it may not extend proximally beyond the proximal end of the blood conduit. In some embodiments, depending on the configuration of the expandable housing for example, it may be desirable to have a portion of impeller 390 or 400 extend proximally beyond a proximal end of the conduit to allow for the at least partially radial flow from the impeller at the outlet.

In any of the embodiments herein that have multiple impellers, impellers may have different configurations, optionally being different in at least one of blade configuration, hub configuration, impeller length, blade length, outermost (radial) dimension, or flow type (e.g., axial vs mixed or radial). Any impeller of a multi-impeller pump may have a different flow type from any other impeller in the pump, wherein any impeller may be a radial flow impeller, an axial flow impeller, or a mixed flow impeller.

The disclosure herein also includes methods of use that include pressurizing and/or inflating one or more inflatable impellers. For example, impellers 390 and 400 may be in fluid communication with a fluid source so that fluid can be advanced through and/or into the impeller to increase the pressure on the impeller and thereby increase the stiffness for use. As set forth herein, fluid pressure may be used to expand the impeller to at least some extent, or it may be used only to increase stiffness even if not for expansion purposes. Fluid pressure may be used to increase the stiffness of the one or more impellers. In use, the impeller may be first deployed from a delivery device (e.g., a sheath). Deployment in this step may cause the impeller hub to expand to at least some extent, and may allow the impeller hub to expand almost to a fully deployed configuration. Deployment may cause the impeller hub to expand only very minimally, if at all. Deployment can include allowing one or more blades to self-expand (e.g., unwrap/uncurl), while a separately expandable region (e.g., hub) may not self-expand at all, or may self-expand to at least some extent. Any expansion method can also include delivering fluid into and/or through the impeller. Fluid inflation may cause the impeller hub to expand to some extent, it may cause the impeller hub to expand a very slight amount, or it may not cause the impeller hub to expand at all. Fluid inflation may include causing the impeller to change configuration initially during an initial period of time, followed by a period of minimal or no configuration change (with the same fluid flow rate as the same time period) but causing an increase in fluid pressure that increases the stiffness of at least a portion of the impeller.

To collapse the impeller for removal, some methods include advancing a re-sheathing device (e.g., sheath) over the pump portion, which at least partially causes the impeller to collapse. Methods of collapse may also include a step that actively removes fluid that may remain inside the inflated impeller. For example, fluid may be pumped out of the fluid inflation lumen prior to or during collapse. Methods of collapse and removal may include both steps. Collapsing the impeller can include radially collapsing one portion of the impeller (e.g., the proximal region), and causing at least a portion of a blade to wrap/curl relative to a long axis of the impeller.

FIG. 15 illustrates a portion of an exemplary catheter blood pump that includes pump portion 420, which may be modified with any suitable feature (component(s) or method of use) herein. Pump portion 420 includes collapsible distal impeller 423 and collapsible proximal impeller 422, which are shown by way of example to have similar configurations to impellers 390 and 400, and may have any suitable feature thereof (e.g., inflatable) or any other impeller herein. Proximal impeller 422 is shown extending partially proximally outside of blood conduit 421 at the outflow, causing at least partially radial flow F as shown. Other aspects of the system are not shown for clarity, such as proximal struts, catheter shaft, motor, purge fluid pathways, etc., but are understood to be incorporated by reference herein. Distal impeller 423 in this example is configured to create some radial flow, as shown with flow “F” and arrows. Expandable impeller housing 421 includes a radially enlarged region, that has a length 424, which creates a channel or passageway for a radial flow component from distal impeller 423. The enlarged bulbous region has a configuration/shape that also causes it to act as a diffuser for the distal impeller. As referenced elsewhere herein, radial impellers demonstrate higher performance, can run at lower RPMs, have higher flow rates, and/or allow for the diameters/size to be smaller. The enlarged region of conduit 421 may also function as a pump portion stabilization and/or centration member, engaging tissue (e.g., ventricular tissue) to help stabilize the pump and/or center the pump relative to the tissue, such as distal to aortic valve in a left ventricle. A proximal impeller, if the pump is a multi-impeller pump, may be different than the distal impeller, such as any other impeller herein.

The entire disclosure in WO2020/073047A1 is included by reference herein for all purposes. For example, disclosure in WO2020/073047A1 related to delivery of fluid from a fluid source external the patient may be incorporated by reference into any embodiment herein in which fluid is delivered through a catheter (in a fluid pathway such as a lumen) to increase pressure and/or inflate an impeller hub.

A complete listing of the claims follows:

1. A catheter blood pump, comprising:
an expandable pump portion extending distally from a catheter, the pump portion including an expandable impeller housing that includes an expandable blood conduit defining a blood lumen, and
a collapsible impeller comprising a collapsible and inflatable hub and one or more blades extending from the hub, the inflatable hub having a ramped surface that is disposed at least partially proximal to a proximal end of the blood conduit, the ramped surface positioned relative to the proximal end of the blood conduit to create a least partial radial flow at an outflow of the pump portion,
the inflatable hub having an internal volume to facilitate fluid delivery to the internal volume to increase fluid pressure within the internal volume of the hub.

2. The blood pump of claim 1, wherein the inflatable hub further comprises a proximal non-bladed section, the proximal non-bladed having an outer profile with a tapered configuration that tapers downward in a proximal direction, the outer profile at least partially facilitating collapse of the inflatable hub.

3. The blood pump of claim 1, wherein the ramped surface is in a bladed region of the inflatable hub.

4. The blood pump of claim 1, wherein the internal volume is greater a deployed configuration than in a collapsed delivery configuration.

5. The blood pump of claim 1, wherein the one or more impeller blades are not in fluid communication with the internal volume.

6. The blood pump of claim 1, wherein the impeller hub comprises at least one material such that the impeller hub is stiffer after the inflation fluid is delivered to the impeller hub.

7. The blood pump of claim 1, wherein the inflatable hub comprises a first section with a first thickness, and a second section with a second thickness different than the first thickness.

8. The blood pump of claim 7, wherein the first section and the second section comprises polymeric material.

9. The blood pump of claim 1, wherein the internal volume is adapted to be in fluid communication with a purge fluid source, the purge fluid source adapted to be disposed outside a patient's body.

10. The blood pump of claim 1, wherein the hub includes a bladed region with a trumpet configuration, the one or more blades disposed in the bladed region.

11. The blood pump of claim 1, wherein the inflatable hub is inflatable in a bladed region and is inflatable in a non-bladed region.

12. The blood pump of claim 11, wherein the internal volume includes a bladed internal volume and a non-bladed internal volume.

13. The blood pump of claim 12, wherein the bladed internal volume is greater than the internal non-bladed volume when the hub is inflated.

14. The blood pump of claim 12, wherein the bladed internal volume is less than the internal non-bladed volume when the hub is inflated.

15. The blood pump of claim 1, wherein the inflatable hub comprises a compliant or semi-compliant material.

16. The blood pump of claim 1, wherein the inflatable hub comprises at least one material such that it self-expands to an at least partially deployed configuration upon release of a sheathing force.

17. The blood pump of claim 16, wherein the inflatable hub deforms from the self-expanded configuration after the fluid delivery.

18. The blood pump of claim 1, wherein at least one surface that is part of the impeller hub is deformable from a collapsed delivery position to a fully deployed configuration in which an outermost dimension of the at least one surface, relative to a long axis of the impeller, is greater in the fully deployed configuration than it is in the collapsed delivery position.

19. The blood pump of claim 18, wherein the at least one surface is configured to at least partially self-expand, and wherein the at least one surface is adapted to be pressurized after at least partial self-expansion upon delivery of the fluid.

20. The blood pump of claim 1, wherein the inflatable hub comprises a plurality of support members adapted and configured to provide radial support to the inflatable hub.

21-58. (canceled)
